(19) United States
(12) Patent Application Publication
(10) Pub. No.: US 2025/0263487 A1
(43) Pub. Date: Aug. 21, 2025

(54) METHODS TO MANIPULATE ALPHA-FETOPROTEIN (AFP)

(71) Applicant: The Brigham and Women's Hospital, Inc., Boston, MA (US)

(72) Inventors: Richard S. BLUMBERG, Waltham, MA (US); Kristi BAKER, Edmonton (CA); Michal PYZIK, Cambridge, MA (US); Amit GANDHI, Billerica, MA (US)

(73) Assignee: The Brigham and Women's Hospital, Inc., Boston, MA (US)

(21) Appl. No.: 18/824,264

(22) Filed: Sep. 4, 2024

Related U.S. Application Data
(63) Continuation of application No. 16/284,005, filed on Feb. 25, 2019, now Pat. No. 12,110,333.
Application No. 16/284,005 is a continuation of application No. 15/306,665, filed on Oct. 25, 2016, now abandoned.
(60) Provisional application No. 62/101,539, filed on Jan. 9, 2015.
(60) Provisional application No. 61/984,252, filed on Apr. 25, 2014.

(51) Int. Cl. C07K 16/28; A61K 39/395; A61K 45/06; C07K 16/44

(52) U.S. Cl. CPC C07K 16/283; A61K 39/3955; A61K 45/06; C07K 16/44; C07K 2317/76; C07K 2317/77

(57) ABSTRACT

As demonstrated herein, soluble human FcRn binds to AFP with affinities greater than observed with albumin, and is able to interfere with FcRn-mediated protection of and functional associations with IgG. Accordingly, provided herein, in some aspects, are compositions and methods to inhibit FcRn and AFP interactions in diseases or disorders where elevated AFP levels are associated with immunosuppression. Also provided herein, in some aspects, are compositions and methods to enhance or potentiate FcRn and AFP interactions in diseases or disorders with decreased AFP levels or diseases or disorders where increasing AFP levels increasing with immunosuppression.

### CROSS-REFERENCE TO RELATED APPLICATION

This application is a Continuation Application of U.S. Ser. No. 16/284,005 filed on Feb. 25, 2019, which is a Continuation Application of U.S. Ser. No. 15/306,665 filed on Oct. 25, 2016, which is a 35 U.S.C. § 371 National Phase Entry Application of International Application No. PCT/US2015/026860 filed Apr. 21, 2015, and which claims benefit under 35 U.S.C. § 119(e) of U.S. Provisional Application No. 61/984,252 filed Apr. 25, 2014, and U.S. Provisional Application No. 62/101,539 filed Jan. 9, 2015, the contents of each of which are incorporated herein by reference in their entireties.

### GOVERNMENT SUPPORT

This invention was made with government support under DK-53056 awarded by the National Institutes of Health (NIH). The government has certain rights in the invention

### SEQUENCE LISTING

The instant application contains a Sequence Listing which has been submitted in XML format via Patent Center and is hereby incorporated by reference in its entirety. Said XML copy, created on Sep. 3, 2024, is named 043214-081613USC3_SL.xml and is 30,371 bytes in size.

### TECHNICAL FIELD

The technical field relates to compositions and methods for modulating alpha-fetoprotein levels and activities.

### BACKGROUND

Alpha-fetoprotein (AFP) is a major plasma protein in the fetus, where it is produced by the yolk sac and liver (Ingram et al., 1981). In an adult, its concentration is very low, except when a tumor, such as a hepatoma or teratoma, is present. The alpha-fetoprotein and albumin genes are syntenic, and mammalian AFP and serum albumin genes are believed to have arisen through duplication of an ancestral gene 300 to 500 million years ago.

### SUMMARY

The compositions and methods described herein are based, in part, on the discovery that alpha-fetoprotein (AFP) is a third ligand for the neonatal Fc receptor. As demonstrated herein, soluble human FcRn binds to AFP with affinities greater than observed with albumin, and is able to interfere with FcRn-mediated protection of and functional associations with IgG. As further shown herein, the AFP binding site on FcRn overlaps with the albumin binding sites on FcRn, and antibodies that are specific for the albumin site on hFcRn can decrease FcRn-mediated AFP transport. As also demonstrated herein, the binding of FcRn to AFP occurs over a much wider pH range than that observed for IgG and albumin, which typically bind under acidic pH conditions. In addition, provided herein are single nucleotide polymorphisms in AFP that can impact binding of AFP with human FcRn, such as, for example, G109R, R487S, and S445L that increase AFP-FcRn binding, and T451I and D536V, that decrease AFP-FcRn binding.

Accordingly, provided herein, in some aspects, are compositions and methods to inhibit FcRn and AFP interactions in diseases or disorders where elevated AFP levels are associated with immunosuppression. Also provided herein, in some aspects, are compositions and methods to enhance or potentiate FcRn and AFP interactions in diseases or disorders with decreased AFP levels or diseases or disorders where AFP levels increase with immunosuppression.

In some aspects, provided herein are pharmaceutical compositions comprising an inhibitor of AFP-FcRn and a pharmaceutically acceptable carrier, wherein said inhibitor of AFP-FcRn inhibits binding between AFP and FcRn.

In some embodiments of these aspects and all such aspects described herein, the inhibitor of AFP-FcRn comprises a T451I and/or D536V polymorphism of wild-type AFP.

In some embodiments of these aspects and all such aspects described herein, the inhibitor of AFP-FcRn inhibits binding between Y521 and/or V522 of AFP and R42 of FcRn.

In some embodiments of these aspects and all such aspects described herein, the inhibitor of AFP-FcRn inhibits binding between P492 of AFP and R69 of FcRn.

In some embodiments of these aspects and all such aspects described herein, the inhibitor of AFP-FcRn inhibits binding between Q441 and/or V493 of AFP and E44 of FcRn.

In some embodiments of these aspects and all such aspects described herein, the inhibitor of AFP-FcRn inhibits binding between H534 and/or E589 of AFP and N173 of FcRn.

In some embodiments of these aspects and all such aspects described herein, the inhibitor of AFP-FcRn inhibits binding between the hydrophobic core of AFP and FcRn.

In some embodiments of these aspects and all such aspects described herein, the inhibitor of AFP-FcRn inhibits binding between L484, V493, V497, and/or F512 of AFP and V57, W59, and/or W61 of FcRn.

In some embodiments of these aspects and all such aspects described herein, the inhibitor of AFP-FcRn inhibits binding between T443 of AFP and E62 and/or W59 of FcRn.

In some embodiments of these aspects and all such aspects described herein, the inhibitor of AFP-FcRn inhibits binding between D529 of AFP and S230 of FcRn.

In some embodiments of these aspects and all such aspects described herein, the inhibitor of AFP-FcRn inhibits binding between S527 and/or D528 of AFP and E50 and/or 67Y of β2m complexed with FcRn.

In some embodiments of these aspects and all such aspects described herein, the inhibitor of AFP-FcRn inhibits binding between R604 of AFP and the carbonyl oxygen at E50 of β2m complexed with FcRn.

In some embodiments of these aspects and all such aspects described herein, the inhibitor of AFP-FcRn inhibits binding between Q597 of AFP and E69 of β2m complexed with FcRn.

In some embodiments of these aspects and all such aspects described herein, the inhibitor of AFP-FcRn inhibits binding between E106 of AFP and H161 of FcRn.

In some embodiments of these aspects and all such aspects described herein, the inhibitor of AFP-FcRn inhibits binding between S135 of AFP and H161 of FcRn.

In some embodiments of these aspects and all such aspects described herein, the inhibitor of AFP-FcRn inhibits binding between F531, F533, F552, and/or F575 of AFP and W53 of FcRn.

In some embodiments of these aspects and all such aspects described herein, the inhibitor of AFP-FcRn is an antibody or antigen-binding fragment thereof, a small molecule compound, or an RNA or DNA aptamer.

In some embodiments of these aspects and all such aspects described herein, the antibody or antigen-binding fragment thereof is a chimeric, humanized, or completely human antibody or antigen-binding fragment thereof.

In some embodiments of these aspects and all such aspects described herein, the inhibitor of AFP-FcRn inhibits or blocks the AFP binding site on FcRn.

Also provided herein, in some aspects, are pharmaceutical compositions comprising an AFP-FcRn potentiator and a pharmaceutically acceptable carrier.

In some embodiments of these aspects and all such aspects described herein, the AFP-FcRn potentiator comprises a G109R, R487S, and/or S445L polymorphism of wild-type AFP that increases AFP-FcRn binding.

In some embodiments of these aspects and all such aspects described herein, the AFP-FcRn potentiator enhances binding between Y521 and/or V522 of AFP and R42 of FcRn.

In some embodiments of these aspects and all such aspects described herein, the AFP-FcRn potentiator enhances binding between P492 of AFP and R69 of FcRn.

In some embodiments of these aspects and all such aspects described herein, the AFP-FcRn potentiator enhances binding between Q441 and/or V493 of AFP and E44 of FcRn.

In some embodiments of these aspects and all such aspects described herein, the AFP-FcRn potentiator enhances binding between H534 and/or E589 of AFP and N173 of FcRn.

In some embodiments of these aspects and all such aspects described herein, the AFP-FcRn potentiator enhances binding between the hydrophobic core of AFP and FcRn.

In some embodiments of these aspects and all such aspects described herein, the AFP-FcRn potentiator enhances binding between L484, V493, V497, and/or F512 of AFP and V57, W59, and/or W61 of FcRn.

In some embodiments of these aspects and all such aspects described herein, the AFP-FcRn potentiator enhances binding between T443 of AFP and E62 and/or W59 of FcRn.

In some embodiments of these aspects and all such aspects described herein, the AFP-FcRn potentiator enhances binding between D529 of AFP and S230 of FcRn.

In some embodiments of these aspects and all such aspects described herein, the AFP-FcRn potentiator enhances binding between S527 and/or D528 of AFP and E50 and/or 67Y of β2m complexed with FcRn.

In some embodiments of these aspects and all such aspects described herein, the AFP-FcRn potentiator enhances binding between R604 of AFP and the carbonyl oxygen at E50 β2m complexed with FcRn.

In some embodiments of these aspects and all such aspects described herein, the AFP-FcRn potentiator enhances binding between Q597 of AFP and E69 of β2m complexed with FcRn.

In some embodiments of these aspects and all such aspects described herein, the AFP-FcRn potentiator enhances binding between E106 of AFP and H161 of FcRn.

In some embodiments of these aspects and all such aspects described herein, the AFP-FcRn potentiator enhances binding between S135 of AFP and H161 of FcRn.

In some embodiments of these aspects and all such aspects described herein, the AFP-FcRn potentiator enhances binding between 531, F533, F552, and/or F575 of AFP and W53 of FcRn.

In some embodiments of these aspects and all such aspects described herein, the AFP-FcRn potentiator is an antibody or antigen-binding fragment thereof, a small molecule compound, an RNA or DNA aptamer, or an AFP functional fragment.

In some embodiments of these aspects and all such aspects described herein, the antibody or antigen-binding fragment thereof is a chimeric, humanized, or completely human antibody or antigen-binding fragment thereof.

In some embodiments of these aspects and all such aspects described herein, the AFP-FcRn potentiator binds FcRn and mimics AFP binding.

In some embodiments of these aspects and all such aspects described herein, the AFP-FcRn potentiator binds or physically interacts with AFP or FcRn, and enhances or promotes interactions between AFP and FcRn.

In some embodiments of these aspects and all such aspects described herein, the AFP-functional fragment comprises Y521 and/or V522 of AFP and can interact with R42 of FcRn.

In some embodiments of these aspects and all such aspects described herein, the AFP-functional fragment comprises P492 of AFP and can interact with R69 of FcRn.

In some embodiments of these aspects and all such aspects described herein, the AFP-functional fragment comprises Q441 and/or V493 of AFP and can interact with E44 of FcRn.

In some embodiments of these aspects and all such aspects described herein, the AFP-functional fragment comprises H534 and/or E589 of AFP and can interact with N173 of FcRn.

In some embodiments of these aspects and all such aspects described herein, the AFP-functional fragment comprises L484, V493, V497, and/or F512 of AFP and can interact with V57, W59, and/or W61 of FcRn.

In some embodiments of these aspects and all such aspects described herein, the AFP-functional fragment comprises T443 of AFP and can interact with E62 and/or W59 of FcRn.

In some embodiments of these aspects and all such aspects described herein, the AFP-functional fragment comprises D529 of AFP and can interact with S230 of FcRn.

In some embodiments of these aspects and all such aspects described herein, the AFP-functional fragment comprises S527 and/or D528 of AFP and can interact with E50 and/or 67Y of β2m complexed with FcRn.

In some embodiments of these aspects and all such aspects described herein, the AFP-functional fragment comprises R604 of AFP and can interact with the carbonyl oxygen at E50 of β2m complexed with FcRn.

In some embodiments of these aspects and all such aspects described herein, the AFP-functional fragment comprises Q597 of AFP and can interact with E69 of β2m complexed with FcRn.

In some embodiments of these aspects and all such aspects described herein, the AFP-functional fragment comprises E106 of AFP and can interact with H161 of FcRn.

In some embodiments of these aspects and all such aspects described herein, the AFP-functional fragment comprises S135 of AFP and can interact with H161 of FcRn.

In some embodiments of these aspects and all such aspects described herein, the AFP-functional fragment comprises F531, F533, F552, and/or F575 of AFP and can interact with W53 of FcRn.

In some embodiments of these aspects and all such aspects described herein, an AFP-functional fragment comprises SEQ ID NO: 4 or AFP (1-575).

In some embodiments of these aspects and all such aspects described herein, an AFP-functional fragment comprises SEQ ID NO: 5 or AFP (484-575).

In some aspects, provided herein are methods to inhibit or reduce FcRn and AFP interactions in a disease or disorder associated with elevated AFP levels comprising ad mistering a therapeutically effective amount of any of the pharmaceutical compositions comprising an AFP-FcRn inhibitor described herein to a subject in need thereof.

In some embodiments of these aspects and all such aspects described herein, the subject has or has been diagnosed with cancer.

In some embodiments of these aspects and all such aspects described herein, the subject has or has been diagnosed with a cancer or tumor of primitive origin, a tumor of liver origin, such as a hepatoma, a tumor of biliary origin, such as cholangiocarcinoma, stomach cancer, pancreatic cancer, or a teratocarcinoma.

In some embodiments of these aspects and all such aspects described herein, the method further comprises administering an anti-cancer therapy or agent to the subject.

In some embodiments of these aspects and all such aspects described herein, the method further comprises administering a tumor or cancer antigen.

In some aspects, provided herein are methods to increase or potentiate FcRn and AFP interactions in diseases or disorders associated with decreased AFP levels, or where increasing AFP levels is beneficial, comprising administering a therapeutically effective amount of any of the pharmaceutical compositions comprising an AFP-FcRn potentiator described herein to a subject in need thereof.

In some embodiments of these aspects and all such aspects described herein, the subject in need is pregnant or is at risk for having a problem with establishing and/or maintaining a pregnancy.

In some embodiments of these aspects and all such aspects described herein, the subject has or has been diagnosed with an autoimmune disease or disorder.

In some embodiments of these aspects and all such aspects described herein, the subject has or has been diagnosed with host versus graft disease (HVGD), is an organ or tissue transplant recipient, or a recipient of an allogenic transplant.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “AFP-FcRn inhibitor” and “alpha fetoprotein and FcRn inhibitor,” “inhibitor of AFP-FcRn,” or “inhibitor of AFP and FcRn interactions” refer to a molecule or agent that significantly blocks, inhibits, reduces, or interferes with the interaction between AFP and FcRn and their resultant biological or functional activity in vitro, in situ, and/or in vivo, including activity of downstream pathways mediated by AFP binding to FcRn and signaling, such as, for example, transcytosis of AFP, inhibition of T cell stimulation by IgG comprising immune complex-primed dendritic cells, AFP-mediated inhibition of immune responses, and/or increased serum half-life of AFP. Exemplary AFP-FcRn inhibitors contemplated for use in the various aspects and embodiments described herein include, but are not limited to, antibodies or antigen-binding fragments thereof that specifically bind to one or more amino acid residues or epitopes on AFP and/or FcRn involved in the binding and/or interactions of AFP and FcRn, and inhibit/reduce/block AFP and FcRn interactions and/or binding; small molecule agents that target or specifically bind one or more amino acid residues on AFP and/or FcRn involved in the binding and/or interactions of AFP and FcRn, and inhibit/reduce/block AFP and FcRn interactions and/or binding; RNA or DNA aptamers that bind to AFP and/or FcRn and inhibit/reduce/block AFP and FcRn interactions and/or binding; and/or AFP fragments or fusion polypeptides thereof that block endogenous AFP interactions with FcRn.

“Decreased/decreasing interaction between AFP and FcRn,” “reduced/reducing interaction between AFP and FcRn,” “inhibits binding,” or “inhibited/inhibiting interaction between AFP and FcRn” as used interchangeably herein, generally means either reducing or inhibiting the interaction between or binding of AFP and FcRn by at least 5%, at least 10%, at least 25%, at least 50%, at least 60%, at least 70%, at least 80%, at least 90%, at least 95%, at least 98%, or more, compared to the interaction between AFP and FcRn under the same conditions but without the presence of AFP-FcRn inhibitors described herein. Assays for measuring such inhibition or reduced interactions are known in the art and are described herein in the Examples.

As used herein, the terms “AFP-FcRn potentiator,” “potentiator of AFP-FcRn interaction,” “AFP-FcRn activator agent,” and “AFP-FcRn agonist agent” refer to a molecule or agent that mimics or up-regulates (e.g., increases, potentiates or supplements) the biological activity of AFP binding to FcRn in vitro, in situ, and/or in vivo, including downstream pathways mediated by AFP binding to FcRn and signaling, such as, for example, transcytosis of AFP, inhibition of T cell stimulation by immune complex-primed dendritic cells, AFP-mediated inhibition of immune responses, and/or increased serum half-life of AFP. An AFP-FcRn potentiator or agonist can be, in some embodiments, an AFP protein fragment or derivative thereof having at least one bioactivity of the wild-type AFP. An AFP-FcRn potentiator can also be a compound which increases the interaction of AFP with FcRn, for example. Exemplary AFP-FcRn potentiators or agonists contemplated for use in the various aspects and embodiments described herein include, but are not limited to, antibodies or antigen-binding fragments thereof that specifically bind to AFP bound to FcRn and enhance the interaction and/or block FcRn binding to albumin and/or IgG but allow binding of AFP to FcRn; RNA or DNA aptamers that bind to FcRn and mimic AFP binding to FcRn; AFP structural analogs or AFP fragment, derivatives, or fusion polypeptides thereof; and small molecule agents that target or bind to FcRn and act as functional mimics of AFP binding to FcRn.

As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The terms “antibody fragment” or “antigen-binding fragment” include: (i) the Fab fragment, having V_{L}, C_{L}, V_{H} and C_{H}1 domains; (ii) the Fab′ fragment, which is a Fab fragment having one or more cysteine residues at the C-terminus of the C_{H}I domain; (iii) the Fd fragment having V_{H} and C_{H}I domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

The term “therapeutically effective amount” therefore refers to an amount of the inhibitors or potentiators described herein, using the methods as disclosed herein, that is sufficient to provide a particular effect when administered to atypical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign tumors and malignant cancers, as well as dormant tumors or micrometastases. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

As used herein, the phrase “at risk for having a problem with establishing and/or maintaining a pregnancy” refers to a subject (e.g., a human) that is predisposed to experiencing a problem with establishing and/or maintaining a pregnancy. This predisposition may be genetic (e.g., a particular genetic tendency to experience a problem with establishing and/or maintaining a pregnancy, such as heritable disorders), or due to other factors (e.g., age, prior experience of a problem with establishing and/or maintaining a pregnancy, drug or alcohol use, environmental conditions, exposures to detrimental compounds present in the environment, etc.). Thus, it is not intended that the present invention be limited to any particular risk, nor is it intended that the present invention be limited to any particular problem with establishing and/or maintaining a pregnancy.

As used herein, an “autoimmune disease” refers to a class of diseases in which a subject's own antibodies react with host tissue or in which immune effector T cells are autoreactive to endogenous self-peptides and cause inflammation and/or destruction of tissue. Thus an immune response is mounted against a subject's own antigens, referred to as self-antigens. A “self-antigen” as used herein refers to an antigen of a normal host tissue. Normal host tissue does not include cancer cells.

### BRIEF DESCRIPTION OF THE FIGURES

FIG. 1 demonstrates that hAFP is transcytosed by hFcRn at acidic and neutral pH. Transcytosis of human AFP in MDCK II cells co-expressing human FcRn and β2microglobulin (hFcRn/β2m) or vector control at pH 6 and 7.4. B→A Basolateral to apical direction, A→B Apical to Basolateral direction.

FIG. 2 demonstrates that hAFP is transcytosed by mouse FcRn. Transcytosis of human AFP in MDCK II cells co-expressing mouse FcRn and β2microglobulin (mFcRn/mβ2m) or vector control at pH 7.4. B→A Basolateral to apical direction, A→B Apical to Basolateral direction.

FIG. 3 demonstrates that AFP transcytosis by hFcRn is blocked by ADM31 antibody, which specifically binds to an epitope on FcRn that contains the albumin binding site. Transcytosis of human AFP in MDCK II cells co-expressing human FcRn and β2microglobulin (mFcRn/mβ2m) or vector control at pH 7.4 in presence of anti-human FcRn antibody (ADM31) or isotype control. B→A Basolateral to apical direction.

FIG. 4 demonstrates that AFP hinders FcRn-mediated transcytosis of IgG. Transcytosis of human IgG in MDCK II cells co-expressing human FcRn and β2microglobulin (hFcRn/hβ2m) or vector control at pH 6 which have been pre-incubated with hAFP or Human Serum Albumin (HSA) as control at pH 7.4. Basolateral to apical direction is shown.

FIG. 5 demonstrates that AFP binds to human and mouse FcRn at neutral pH. SPR analyses of hAFP binding to hFcRn (left panel) or mFcRn (right panel) at neutral pH.

FIG. 6 demonstrates that AFP binds to hFcRn at acidic pH. SPR analyses of hAFP binding to hFcRn at pH 6.

FIG. 7 demonstrates that AFP inhibits T cell stimulation by IgG-immune complex (IC) primed dendritic cells (DC). hAFP blocks proliferation (IL-2 secretion) of CD8+(OT-I, left panel) or CD4+(OT-II, right panel) T cells in response to antigen in IgG-IC from bone marrow (BM). DC from hFCGRT/hB2M/mFcgrt−/− human FcRn and β2-microglobulin transgenic and mouse FcRn knockout) mice were treated with 100 μg/ml of IgG or IHH-IgG (FcRn-defective IgG) in association with 0, 0.5, 1, or 5 μg/ml of OVA in presence of 100 μg/ml of hAFP and then co-cultured with either OVA-specific CD8+ or CD4+ T cells. 24 after the stimulation IL-2 secretion in the supernatants were measured by ELISA.

FIG. 8 demonstrates that ADM31 blocks AFP-FcRn-mediated inhibitory functions. ADM31 α-hFcRn monoclonal antibody blocks hAFP inhibition of CD8+ T cell IL-2 secretion in response to antigen in IgG-IC. BMDC from hFCGRT/hB2M/mFcgrt−/− mice were treated with 100 μg/ml of IgG or IHH-IgG in association with 0.5 μg/ml OVA in presence of 50 μg/ml of hAFP or HSA and 50 μg/ml of ADM31 or isotype control, and then co-cultured with OVA-specific CD8+ T cells. 24 after the stimulation IL-2 secretion in the supernatants were measured by ELISA.

FIG. 9 demonstrates that ADM31 blocks AFP-FcRn-mediated inhibitory functions. ADM31 α-hFcRn monoclonal antibody blocks hAFP inhibition of CD8+ T cell proliferation in response to antigen in IgG-IC. BMDC from hFCGRT/hB2M/mFcgrt−/− mice were treated with 100 μg/ml of IgG or IHH-IgG in association with 0.5 μg/ml OVA in presence of 50 μg/ml of hAFP or HSA and 50 μg/ml of ADM31 or isotype control, and then co-cultured with CD8+ T cells labelled with eFluor670 Proliferation Dye. 72 hrs later the cells were acquired. Percent of proliferated cells is displayed.

FIG. 10 demonstrates that administration of hAFP results in increased clearance of hIgG antibodies from systemic circulation. hFCGRT/hB2M/mFcgrt−/− mice were injected with hIgG and the following day with hAFP. 24, 48 and 72 hrs later blood samples were collected and the amount of hIgG was quantified by ELISA and compared to Day 0. The results illustrate that AFP injection resulted in faster clearance of hIgG from circulation.

FIG. 11 shows an AFP homology model derived from human serum albumin (HSA) Crystal Structure (PDB ID: 4N0F). Based on high homology between HSA and AFP, a structural model of AFP was built and superimposed on FcRn:HSA:Fc-YTE structure (PDB ID 4N0U) with RMSD of 0.072. All the figures were drawn using PyMOL (DELANO SCIENTIFIC) and labels were added using ADOBE® Photoshop.

FIG. 12 depicts superimposition of AFP model on HSA (left panel) or FcRn-HSA-IgG ternary complex crystal structure (PDB ID: 4N0U) (right panel).

FIG. 13 depicts HSA Y497/V498 residues are conserved in AFP (Y521/V522) and interact with FcRn R42. HSA/AFP have conserved residues in Domain III that establish binding to FcRn.

FIG. 14 demonstrates that HSA P468 residue is conserved in AFP (P492) and interacts with FcRn R69. HSA/AFP conserved residues in Domain III that establish binding to FcRn

FIG. 15 demonstrates that HSA Q417/V469 residues are conserved in AFP (Q441/V493) and interact with FcRn E44. HSA/AFP conserved residues in Domain III establish binding to FcRn AFP. HSA V469/AFPV493 make backbone contacts with conserved HSA H464/AFP H488.

FIG. 16 demonstrates that HSA H510/E565 residues are conserved in AFP (H534/E589) and interact with FcRn N173. HSA/AFP conserved residues in Domain III establish binding to FcRn.

FIG. 17 demonstrates that hydrophobic core centered on HSA L460/V469/V473/F488 is conserved in AFP (L484/V493/V497/F512) and interacts with FcRn V57/W59/W61. HSA/AFP conserved residues in Domain III establish binding to FcRn.

FIG. 18 demonstrates that HSA S419 residue is not conserved in AFP (T443) yet is able to interact with FcRn E62/W59. HSA/AFP non-conserved residues in Domain III preserve AFP binding to FcRn.

FIG. 19 demonstrates that HSA E505 non-conserved residue in AFP (D529) preserves binding to FcRn S230. HSA/AFP non-conserved residues in Domain III preserve AFP binding to FcRn.

FIG. 20 demonstrates that AFP S527/D528 residues make contacts with β2m E50 and 67Y that are not present in HSA (N503, A504) providing new interactions. HSA/AFP non-conserved residues that increase AFP binding to FcRn through new contacts with β2m and is not pH dependent.

FIG. 21 demonstrates that AFP R604 makes additional contacts with β2m E50, providing new interactions. HSA/AFP non-conserved residues increase AFP binding to β2m. HSA Q580 lacks these interactions.

FIG. 22 demonstrates that AFP Q597 residue is better positioned to make contacts with β2m E69 providing stronger interaction. HSA/AFP non-conserved residues establish new and increased AFP-β2m interactions. HSA K573 lacks these interactions.

FIG. 23 demonstrates that AFP (E106) conserved residue (with HSA E82) makes long range interaction with FcRn H161. Conserved HSA/AFP residues in Domain that interact with FcRn.

FIG. 24 demonstrates that AFP S135 allows AFP interface to come closer to FcRn and makes ˜3 Å interactions with FcRn H161, which is absent in HSA. AFP Domain I-FcRn interaction indicates neutral pH binding. Nearby conserved proline in HSA/AFP occupy same space in interface.

FIG. 25 demonstrates that a substantially conserved hydrophobic core in AFP (F531/F533/F552/F575) centered on FcRn W53: AFP F552 results in stronger AFP-FcRn interactions than HSA A528. AFP-FcRn interactions are consistent with neutral pH binding. AFP (F531/F533/F552/F575) vs HSA (F507/F509/A528/F551)

### DETAILED DESCRIPTION

Compositions and methods are provided herein that relate to the discoveries described herein that alpha fetal protein (AFP) is a third ligand for the neonatal Fc receptor or FcRn.

### FcRn and Alpha Fetoprotein

FcRn, also known as the neonatal Fc receptor, is encoded by the Fcgrt gene. It is a MHC class I-like transmembrane protein consisting of a heavy chain containing three extracellular domains (α1, α2 and α3), a single pass transmembrane domain and a short cytoplasmatic tail (Burmeister et al., 1994a,b; Martin et al., 2001). For proper function, the FcRn heavy chain non-covalently associates with the common β2-microglobulin subunit as a light chain, which interacts with FcRn via residues on the underside of the α1-α2 platform and the side of the α3 domain (West & Bjorkman, 2000). Although the tertiary structure resembles MHC class I molecules with which it shares 22-29% sequence homology (Simister & Mostov, 1989), the mouse and human FcRn genes are located outside the MHC locus, on chromosomes 7 and 19, respectively (Ahouse et al., 1993; Kandil et al., 1996). In further divergence from classical MHC molecules, the sites where peptide residues bind to MHC class I molecules are occluded in FcRn by an arginine side chain and a proline residue, so that FcRn does not present peptide antigens to T-cells (Burmeister et al., 1994a,b).

Most serum proteins have a short serum half-life (about 1-2 days). However, two types of serum proteins, namely albumin and antibodies of the IgG class, have greatly extended serum half-lives. For example, most subclasses of IgG have a half-life of about 10-20 days in humans. The Fc region of IgG is required for this extension of half-life. Thus, truncated IgG polypeptides carrying only the Fc region, and potentially also proteins carrying a short FcRn binding peptide sequence (FcBP) (Sockolosky et al. Proc Natl Acad Sci USA 2012, 109, 16095-100), also show such extended serum half-life. Moreover, when the Fc region is fused with a fusion partner (e.g., a biologically active protein), this Fc fusion protein shows an extended serum half-life due to its interaction with FcRn.

The mechanism by which FcRn extends the serum half-life of IgG and IgG Fc fusion proteins is well established (Ghetie and Ward, 2000, 2002; Roopenian and Akilesh, 2007). FcRn is localized in the endosomal compartments of many cell types, including vascular endothelium. Serum proteins are constantly being endocytosed and directed to the early endosomal vesicles. FcRn is harbored primarily in this acidified vesicle. In this acidified environment, the Fc region binds FcRn, and the IgG/FcRn complex is then recycled either apically or basolaterally back to the plasma membrane, whereupon exposure to the neutral pH 7.2 extracellular environment results in its release into the circulation. In contrast, other endocytosed proteins that do not bind FcRn are not rescued, and thus continue through the endosomal route to catabolic elimination, resulting in their short half-life. The biochemical mechanism by which the Fc region of IgG binds FcRn in an acidic environment is understood. The CH2-CH3-hinge region of the Fc region contains solvent exposed histidine residues, which when protonated, engage residues on FcRn with sufficient affinity to permit IgG to exploit the FcRn recycling pathway to escape catabolic elimination.

Between different species, FcRn exhibits considerable structural variations, which most likely account for the molecule's different ligand binding specificity and slight variations in its functions. The peptide sequences of rat and mouse FcRn, for example, are 91% homologous (Ahouse et al., 1993), whereas the extracellular region of human FcRn shares only 65% amino acid sequence identity with rat FcRn (Story et al., 1994). Bovine FcRn, on the other hand, displays 77% homology to its human counterpart, but exhibits further divergence from rodent FcRn (Kacskovics et al., 2000). Similarly, although mouse and rat FcRn exhibit promiscuous binding to multiple different species of IgG such as horse, rabbit and human, human FcRn binding is significantly more restricted and limited to itself and rabbit (Ober et al., 2001).

Elucidation of the crystal structure revealed that two FcRn molecules bind to a single IgG in a 2:1 stoichiometry (Huber et al., 1993; Sanchez et al., 1999; Schuck et al., 1999). Each IgG heavy chain contains three constant regions (Huber et al., 1976) with one of the FcRn molecules binding to the CH2-CH3 interface of the IgG Fc region (Huber et al., 1993; Sanchez et al., 1999; Schuck et al., 1999; West & Bjorkman, 2000). Such binding between IgG and FcRn occurs in a strictly pH-dependent manner with low micro- to nanomolar affinity at pH<6.5 but no binding at pH 7.5 (Raghavan et al., 1995). Several amino acids on both molecules have been identified to be critical for this interaction. Site-directed mutagenesis approaches have revealed that the residues Ile253, His310 and His435 of IgG play a central role in the interaction with FcRn, as shown within different species (mouse, human and rat) as well as for interspecies binding (Firan et al., 2001; Kim et al., 1994, 1999; Martin et al., 2001; Medesan et al., 1997; Raghavan et al., 1995; Shields et al., 2001). The pKa of His is 6.0-6.5 such that several histidine residues of IgG become protonated below physiologic pH, allowing for the formation of salt bridges with acidic residues on FcRn which in doing so provides the structural basis for the strict pH dependency of IgG-FcRn interactions.

As initially identified in the interaction between rat FcRn and rat IgG2a, residues on FcRn involved in binding IgG include Glu117, Glu118, Glu132, Trp133, Glu135 and Asp137 on the α2 helix (Martin et al., 2001). Although these residues are generally conserved between different species and the main tertiary structure of FcRn with three extracellular ligand-binding domains is preserved, differences between rodent and human FcRn have been described at specific residues and contribute to IgG binding (Vaughn et al., 1997). While human FcRn contains only a single N-glycan moiety in its α2 domain, rat FcRn possesses three additional N-glycan moieties in the α1, α2 and α3 domains (Ahouse et al., 1993; Kuo et al., 2009; Martin et al., 2001; West & Bjorkman, 2000). The Asn128 residue in the α2 domain of rat FcRn, which is lacking in human FcRn, binds to IgG forming a functional “carbohydrate handshake” (Martin et al., 2001; Vaughn & Bjorkman, 1998). In another example, human FcRn displays very limited interspecies IgG binding, extending only to rabbit IgG (Ober et al., 2001), whereas human IgG can bind to cynomolgus FcRn (Bitonti et al., 2004; Dall'Acqua et al., 2006; Zalevsky et al., 2010). Cynomolgus and human IgG have been demonstrated to bind equally well to cynomolgus monkey FcRn (Dall'Acqua et al., 2006), thereby further strengthening the evolutionary significance of the interaction between the Fc region and FcRn. Rodent FcRn, however, is known to be promiscuous by binding to IgG molecules from a variety of species including human, rabbit and bovine IgG as discussed above (Ober et al., 2001). Murinization of human FcRn by mutating the poorly conserved Leu137 residue within the α2 domain of human FcRn to the murine counterpart (glutamic acid) confers binding of human FcRn to mouse IgG1 and IgG2a while reducing binding to human IgG1 twofold (Zhou et al., 2003). The L137E mutation demonstrates that single docking topologies are vitally important in the binding of FcRn to IgG. Apart from the residues discussed above, Ile1 on β2m contributes to IgG binding, most likely by interacting with hydrophobic residues at position 309 of the IgG-Fc domain.

Accordingly, the term “FcRn” as used herein, refers to the molecule comprising the 365 amino acid FcRn large subunit p51 precursor having the amino acid sequence of: MGVPRPQPWALGLLLFLLPGSLGAESHLSLLYHLTAVSSPAPGTPAFWVSGWLGPQQYLS YNSLRGEAEPCGAWVWENQVSWYWEKETTDLRIKEKLFLEAFKALGGKGPYTLQGLLGCE LGPDNTSVPTAKFALNGEEFMNFDLKQGTWGGDWPEALAISQRWQQQDKAANKELTFLLF SCPHRLREHLERGRGNLEWKEPPSMRLKARPSSPGFSVLTCSAFSFYPPELQLRFLRNGL AAGTGQGDFGPNSDGSFHASSSLTVKSGDEHHYCCIVQHAGLAQPLRVELESPAKSSVLV VGIVIGVLLLTAAAVGGALLWRRMRSGLPAPWISLRGDDTGVLLPTPGEAQDADLKDVNV IPATA, (SEQ ID NO: 1), as described by, e.g., NP_001129491.1 or NP_004098.1, which non-covalently associates with the β2 microgobulin (“β2m”) chain having the amino acid sequence of: MSRSVALAVLALLSLSGLEAIQRTPKIQVYSRHPAENGKSNFLNCYVSGFHPSDIEVDLL KNGERIEKVEHSDLSFSKDWSFYLLYYTEFTPTEKDEYACRVNHVTLSQPKIVKWDRDM (SEQ ID NO: 2), as described by, e.g., NP_004039.1, together with any naturally occurring allelic, splice variants, and processed forms thereof. Typically, FcRn refers to human FcRn. The term “FcRn” is also used to refer to truncated forms or fragments of the FcRn polypeptide that retains an FcRn function or activity of interest as described herein, such as, for example, binding to AFP. Reference to any such forms of FcRn can be identified in the application, e.g., by “FcRn (24-110).” Specific residues of FcRn can be referred to as, for example, “FcRn(53) or “W53 of FcRn,” or “E69 of β2m of FcRn.”

As described herein, the inventors have discovered that a third ligand for FcRn is alpha fetoprotein (AFP). As demonstrated herein, human AFP is transcytosed by FcRn at both acidic and neutral pHs, and increasing amounts of human AFP can inhibit FcRn-mediated transcytosis of IgG, and results in increased IgG clearance from systemic circulation, as well as decreased T cell stimulation by IgG immune complexes.

Alpha-fetoprotein (AFP) is a major plasma protein in the fetus, where it is produced by the yolk sac and liver (Ingram et al., 1981). In an adult, its concentration is very low, except when a tumor, such as a hepatoma or teratoma is present. The alpha-fetoprotein and albumin genes are syntenic, and mammalian AFP and serum albumin genes are believed to have arisen through duplication of an ancestral gene 300 to 500 million years ago. After birth, AFP is down-regulated thousands of fold, such that it is not expressed at high levels in a host under homeostatic conditions. It can become subsequently elevated and expressed at high levels during processes associated with particular types of pathology, such as cancers, particularly in tumors of liver origin (e.g., hepatoma), tumors of the biliary system (e.g., cholangiocarcinoma), and in tumors of primitive origin and that are poorly differentiated, such as teratocarcinomas. In addition, elevated AFP levels can occur during chronic liver inflammatory processes, liver regeneration, and during immune activation, such as allogeneic responses.

Accordingly, the term “AFP” as used herein, refers to the 609 amino acid polypeptide having the amino acid sequence of: MKWVESIFLIFLLNFTESRTLHRNEYGIASILDSYQCTAEISLADLATIFFAQFVQEATY KEVSKMVKDALTAIEKPTGDEQSSGCLENQLPAFLEELCHEKEILEKYGHSDCCSQSEEG RHNCFLAHKKPTPASIPLFQVPEPVTSCEAYEEDRETFMNKFIYEIARRHPFLYAPTILL WAARYDKIIPSCCKAENAVECFQTKAATVTKELRESSLLNQHACAVMKNFGTRTFQAITV TKLSQKFTKVNFTEIQKLVLDVAHVHEHCCRGDVLDCLQDGEKIMSYICSQQDTLSNKIT ECCKLTTLERGQCIIHAENDEKPEGLSPNLNRFLGDRDFNQFSSGEKNIFLASFVHEYSR RHPQLAVSVILRVAKGYQELLEKCFQTENPLECQDKGEEELQKYIQESQALAKRSCGLFQ KLGEYYLQNAFLVAYTKKAPQLTSSELMAITRKMAATAATCCQLSEDKLLACGEGAADII IGHLCIRHEMTPVNPGVGQCCTSSYANRRPCFSSLVVDETYVPPAFSDDKFIFHKDLCQA QGVALQTMKQEFLINLVKQKPQITEEQLEAVIADFSGLLEKCCQGQEQEVCFAEEGQKLI SKTRAALGV (SEQ ID NO: 3), as described by, e.g., NP_001125.1, together with any naturally occurring allelic, splice variants, and processed forms thereof. Typically, AFP refers to human AFP. The term “AFP” can also, in some embodiments, be used to refer to truncated forms or fragments of the AFP polypeptide that retain an AFP function or activity of interest as described herein, such as, for example, binding to FcRn. Reference to any such forms of AFP can be identified in the application, e.g., by “AFP (211-402).” Specific residues of AFP can be referred to as, for example, “AFP(531) or “F531 of AFP.”

The discovery as described herein that AFP is a third ligand for FcRn provides novel compositions and methods for the treatment of conditions in which modulating the level of AFP is therapeutic.

### Inhibitors and Potentiators of AFP-FcRn Interactions

Provided herein are compositions and methods thereof based, in part, on the discovery that alpha-fetoprotein (AFP) is a third ligand for the neonatal Fc receptor. As demonstrated herein, soluble human FcRn binds to AFP with affinities greater than observed with albumin, and less than that of IgG. As further shown herein, the AFP binding site on FcRn overlaps directly with both the albumin binding sites on FcRn binding sites on FcRn and indirectly with the IgG binding sites mainly through interactions with β2-microglobulin. IgG interactions with FcRn include amino acid contact sites within β2-microglobulin. Antibodies that are specific for the albumin site on hFcRn can decrease FcRn-mediated AFP transport. As also demonstrated herein, the binding of FcRn to AFP occurs over a much wider pH range than that observed for IgG and albumin, which typically bind under acidic pH conditions. In addition, provided herein are single nucleotide polymorphisms in AFP that can impact binding of AFP with human FcRn, such as, for example, G109R, R487S, and S445L that increase AFP-FcRn binding, and T451I and D536V, that decrease AFP-FcRn binding.

Accordingly, provided herein, in some aspects, are compositions and methods to inhibit or reduce FcRn and AFP interactions in diseases or disorders where elevated AFP levels are associated with immunosuppression. Also provided herein, in some aspects, are compositions and methods thereof to enhance or potentiate FcRn and AFP interactions in diseases or disorders with decreased AFP levels or diseases or disorders where increasing AFP levels is therapeutic, such as subjects in need of increasing immunosuppression.

In some aspects, provided herein are compositions, such as pharmaceutical compositions, comprising inhibitors of AFP-FcRn. Such inhibitors are used to inhibit/block the interaction between AFP and FcRn and/or reduce transcytosis of human AFP, and/or reduce serum half-life of AFP. In particular, in some embodiments of the aspects described herein, such AFP-FcRn inhibitors can be used to inhibit or block the AFP binding site on FcRn, which overlaps with the albumin binding sites on FcRn.

In some embodiments of these aspects and all such aspects described herein, an inhibitor of AFP-FcRn comprises a T451I and/or D536V polymorphism of wild-type AFP that decreases AFP-FcRn binding.

In some embodiments of these aspects and all such aspects described herein, an inhibitor of AFP-FcRn inhibits binding and/or interactions between Y521 and/or V522 of AFP and R42 of FcRn.

In some embodiments of these aspects and all such aspects described herein, an inhibitor of AFP-FcRn inhibits binding and/or interactions between P492 of AFP and R69 of FcRn.

In some embodiments of these aspects and all such aspects described herein, an inhibitor of AFP-FcRn inhibits binding and/or interactions between Q441 and/or V493 of AFP and E44 of FcRn.

In some embodiments of these aspects and all such aspects described herein, an inhibitor of AFP-FcRn inhibits binding and/or interactions between H534 and/or E589 of AFP and N173 of FcRn.

In some embodiments of these aspects and all such aspects described herein, an inhibitor of AFP-FcRn inhibits binding between the hydrophobic core of AFP and FcRn. In some such embodiments, an inhibitor of AFP-FcRn inhibits binding and/or interactions between L484, V493, V497, and/or F512 of AFP and V57, W59, and/or W61 of FcRn.

In some embodiments of these aspects and all such aspects described herein, an inhibitor of AFP-FcRn inhibits binding and/or interactions between T443 of AFP and E62 and/or W59 of FcRn.

In some embodiments of these aspects and all such aspects described herein, an inhibitor of AFP-FcRn inhibits binding and/or interactions between D529 of AFP and S230 of FcRn.

In some embodiments of these aspects and all such aspects described herein, an inhibitor of AFP-FcRn inhibits binding between S527 and/or D528 of AFP and E50 and/or 67Y of β2m complexed with FcRn.

In some embodiments of these aspects and all such aspects described herein, an inhibitor of AFP-FcRn inhibits binding between R604 of AFP and the carbonyl oxygen at E50 of β2m complexed with FcRn.

In some embodiments of these aspects and all such aspects described herein, an inhibitor of AFP-FcRn inhibits binding and/or interactions between Q597 of AFP and E69 of β2m complexed with FcRn.

In some embodiments of these aspects and all such aspects described herein, an inhibitor of AFP-FcRn inhibits binding and/or interactions between E106 of AFP and H161 of FcRn.

In some embodiments of these aspects and all such aspects described herein, an inhibitor of AFP-FcRn inhibits binding and/or interactions between S135 of AFP and H161 of FcRn.

In some embodiments of these aspects and all such aspects described herein, an inhibitor of AFP-FcRn inhibits binding and/or interactions between F531, F533, F552, and/or F575 of AFP and W53 of FcRn.

As used herein, the terms “AFP-FcRn inhibitor” and “alpha fetoprotein and FcRn inhibitor,” “inhibitor of AFP-FcRn,” or “inhibitor of AFP and FcRn interactions” refer to a molecule or agent that significantly blocks, inhibits, reduces, or interferes with the interaction between AFP and FcRn and their resultant biological or functional activity in vitro, in situ, and/or in vivo, including activity of downstream pathways mediated by AFP binding to FcRn and signaling, such as, for example, transcytosis of AFP, inhibition of T cell stimulation by immune complex-primed dendritic cells, AFP-mediated inhibition of immune responses, and/or increased serum half-life of AFP. Exemplary AFP-FcRn inhibitors contemplated for use in the various aspects and embodiments described herein include, but are not limited to, antibodies or antigen-binding fragments thereof that specifically bind to one or more amino acid residues or epitopes on AFP and/or FcRn involved in the binding and/or interactions of AFP and FcRn, and inhibit/reduce/block AFP and FcRn interactions and/or binding; small molecule agents that target or specifically bind one or more amino acid residues on AFP and/or FcRn involved in the binding and/or interactions of AFP and FcRn, and inhibit/reduce/block AFP and FcRn interactions and/or binding; RNA or DNA aptamers that bind to AFP and/or FcRn and inhibit/reduce/block AFP and FcRn interactions and/or binding; and/or AFP fragments or fusion polypeptides thereof that block endogenous AFP interactions with FcRn.

As used herein, an AFP-FcRn inhibitor has the ability to reduce or decrease the interaction between AFP and FcRn and/or their resultant biological or functional activity in vitro, in situ, and/or in vivo by at least 5%, at least 10%, at least 20%, at least 30%, at least 40%, at least 50%, at least 60%, at least 70%, at least 80%, at least 90%, at least 95%, at least 98%, at least 99%, or more, relative to the interaction and/or activity in the absence of the AFP-FcRn inhibitor.

“Decreased/decreasing interaction between AFP and FcRn,” “reduced/reducing interaction between AFP and FcRn,” “inhibits binding,” or “inhibited/inhibiting interaction between AFP and FcRn” as used interchangeably herein, generally means either reducing or inhibiting the interaction between or binding of AFP and FcRn by at least 5%, at least 10%, at least 25%, at least 50%, at least 60%, at least 70%, at least 80%, at least 90%, at least 95%, at least 98%, or more, compared to the interaction between AFP and FcRn under the same conditions but without the presence of AFP-FcRn inhibitors described herein.

In some embodiments of the compositions, methods, and uses described herein, the AFP-FcRn inhibitor is an antibody or antigen-binding fragment thereof. In some embodiments of the aspects described herein, such AFP-FcRn inhibitors can be used to inhibit or block the AFP binding site on FcRn, which overlaps with the albumin binding sites on FcRn, as described herein. In some embodiments, an antibody or antigen-binding fragment inhibitor of AFP-FcRn binds to an epitope that comprises the AFP binding site on FcRn.

In some embodiments of these aspects and all such aspects described herein, the antibody or antigen-binding fragment inhibitor of AFP-FcRn inhibits binding and/or interactions between Y521 and/or V522 of AFP and R42 of FcRn.

In some embodiments of these aspects and all such aspects described herein, the antibody or antigen-binding fragment inhibitor of AFP-FcRn inhibits binding and/or interactions between P492 of AFP and R69 of FcRn.

In some embodiments of these aspects and all such aspects described herein, the antibody or antigen-binding fragment inhibitor of AFP-FcRn inhibits binding and/or interactions between Q441 and/or V493 of AFP and E44 of FcRn.

In some embodiments of these aspects and all such aspects described herein, the antibody or antigen-binding fragment inhibitor of AFP-FcRn inhibits binding and/or interactions between H534 and/or E589 of AFP and N173 of FcRn.

In some embodiments of these aspects and all such aspects described herein, the antibody or antigen-binding fragment inhibitor of AFP-FcRn inhibits binding between the hydrophobic core of AFP and FcRn. In some such embodiments, the antibody or antigen-binding fragment inhibitor of AFP-FcRn inhibits binding and/or interactions between L484, V493, V497, and/or F512 of AFP and V57, W59, and/or W61 of FcRn.

In some embodiments of these aspects and all such aspects described herein, the antibody or antigen-binding fragment inhibitor of AFP-FcRn inhibits binding and/or interactions between T443 of AFP and E62 and/or W59 of FcRn.

In some embodiments of these aspects and all such aspects described herein, the antibody or antigen-binding fragment inhibitor of AFP-FcRn inhibits binding and/or interactions between D529 of AFP and S230 of FcRn.

In some embodiments of these aspects and all such aspects described herein, the antibody or antigen-binding fragment inhibitor of AFP-FcRn inhibits binding between S527 and/or D528 of AFP and E50 and/or 67Y of β2m complexed with FcRn.

In some embodiments of these aspects and all such aspects described herein, the antibody or antigen-binding fragment inhibitor of AFP-FcRn inhibits binding between R604 of AFP and the carbonyl oxygen at E50 of β2m complexed with FcRn.

In some embodiments of these aspects and all such aspects described herein, the antibody or antigen-binding fragment inhibitor of AFP-FcRn inhibits binding and/or interactions between Q597 of AFP and E69 of β2m complexed with FcRn.

In some embodiments of these aspects and all such aspects described herein, the antibody or antigen-binding fragment inhibitor of AFP-FcRn inhibits binding and/or interactions between E106 of AFP and H161 of FcRn.

In some embodiments of these aspects and all such aspects described herein, the antibody or antigen-binding fragment inhibitor of AFP-FcRn inhibits binding and/or interactions between S135 of AFP and H161 of FcRn.

In some embodiments of these aspects and all such aspects described herein, the antibody or antigen-binding fragment inhibitor of AFP-FcRn inhibits binding and/or interactions between F531, F533, F552, and/or F575 of AFP and W53 of FcRn.

Antibodies or antigen-binding fragments thereof that are specific for or that selectively bind AFP, FcRn, and/or AFP bound to FcRn, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Examples of antibody fragments encompassed by the terms antibody fragment or antigen-binding fragment as described herein include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}I domains and one or more cysteine residues at the C-terminus of the C_{H}1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

With respect to a target or antigen, the term “ligand interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen, i.e., AFP, FcRn, or AFP bound to FcRn. For example, in some embodiments, a ligand interaction site on FcRn can be any site to which IgG binds or interacts, or any site to which albumin binds or interacts, or any site to which AFP binds or interacts or which when interacting with AFP affects the conformation of the binding sites for albumin and/or IgG within the FcRn/β2-microglobulin heterodimeric complex. More generally, a “ligand interaction site” can be any site, epitope, antigenic determinant, part, domain or stretch of amino acid residues on a target or antigen to which a binding site of an AFP-FcRn inhibitor described herein can bind such that the interaction or binding between AFP and FcRn (and/or any pathway, interaction, signalling, biological mechanism or biological effect mediated by AFP binding to FcRn is involved) is modulated.

In the context of an antibody or antigen-binding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10.000 times or more better than the affinity with which said amino acid sequence or polypeptide binds to another target or polypeptide. Preferably, when an antibody or antigen-binding fragment thereof is “specific for” a target or antigen, compared to another target or antigen, it is directed against said target or antigen, but not directed against another target or antigen.

However, as understood by one of ordinary skill in the art, in some embodiments, where a binding site on a target is shared or partially shared by multiple, different ligands, an antibody or antigen binding fragment thereof can specifically bind to a target, such as FcRn, and have the functional effect of inhibiting/preventing binding of multiple, different ligands, such as AFP, albumin, and/or IgG. For example, as demonstrated herein, the ADM31 antibody inhibits AFP binding to FcRn, as well as binding of albumin to FcRn (Sand, K. M., et B. Dalhus, G. J. Christianson, M. Bern, S. Foss, J. Cameron, D. Sleep, M. Bjoras, D. C. Roopenian, I. Sandlie and J. T. Andersen (2014). “Dissection of the neonatal Fc receptor (FcRn)-albumin interface using mutagenesis and anti-FcRn albumin-blocking antibodies.” J Biol Chem 289(24): 17228-17239).

Avidity is the measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on a AFP-FcRn inhibitor antibody or antigen-binding fragment thereof described herein will bind to AFP and/or FcRn with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

In some embodiments of the compositions, methods, and uses described herein, the AFP-FcRn inhibitor is a monoclonal antibody.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each antibody in a monoclonal preparation is directed against the same, single determinant on the antigen. It is to be understood that the term “monoclonal antibody” refers to an antibody that is derived from a single clone, including any eukaryotic, prokaryotic, or phage clone, and not the method by which it is produced. The term “monoclonal antibody” as used herein is not limited to antibodies produced through hybridoma technology, and the modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or later adaptations thereof, or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

In some embodiments of the compositions, methods, and uses described herein, the AFP-FcRn inhibitor is a chimeric antibody derivative of an antibody or antigen-binding fragment thereof that binds AFP, FcRn, and/or AFP bound to FcRn.

As used herein, the term“chimeric antibody” refers to an antibody molecule in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the desired antigen, e.g., AFP and/or FcRn. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

In some embodiments of the compositions, methods, and uses described herein, the AFP-FcRn inhibitor is a humanized antibody derivative of an antagonist antibody or antigen-binding fragment thereof that binds AFP, FcRn, and/or AFP bound to FcRn.

Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments of the compositions, methods, and uses comprising any of the AFP-FcRn inhibitor antibodies or antigen-binding fragments thereof described herein, the AFP-FcRn inhibitor antibody or antigen-binding fragment is an antibody derivative. For example, but not by way of limitation, antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids, or alternative scaffolds such as centyrins, DARPINS, or fynomers engineered to bind FcRn and inhibit AFP.

The AFP-FcRn inhibitor antibodies and antigen-binding fragments thereof described herein can be generated by any suitable method known in the art. Monoclonal and polyclonal antibodies against, for example, FcRn, are known in the art. To the extent necessary, e.g., to generate antibodies with particular characteristics or epitope specificity, the skilled artisan can generate new monoclonal or polyclonal AFP-FcRn inhibitor antibodies as briefly discussed herein or as known in the art.

Polyclonal antibodies can be produced by various procedures well known in the art. For example, AFP, FcRn, or fragments thereof comprising one or more of the AFP and/or FcRn interaction sites, can be administered to various host animals including, but not limited to, rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R¹N═C═NR, where R and R¹ are different alkyl groups. Various other adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Suitable adjuvants are also well known to one of skill in the art.

Monoclonal antibodies can be prepared using a wide variety of techniques known in the art including the use of hybridoma, recombinant, and phage display technologies, or a combination thereof. Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

In some embodiments of the compositions, methods, and uses described herein, completely human antibodies are used as AFP-FcRn inhibitors, which are particularly desirable for the therapeutic treatment of human patients.

Human antibodies can be made by a variety of methods known in the art, including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992), the contents of which are herein incorporated by reference in their entireties. Alternatively, phage display technology (McCafferty et al., Nature 348:552-553 (1990)) can be used to produce human antibodies and antibody fragments in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903). [00174]“An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the human heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol. Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., AFP, FcRn, and/or AFP bound to FcRn, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The “Fab” fragment contains a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

Various techniques have been developed for the production of antibody or antigen-binding fragments. The antibodies described herein can be fragmented using conventional techniques and the fragments screened for utility in the same manner as described above for the whole antibodies. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). For example, Fab and F(ab′)₂ fragments of the bispecific and multispecific antibodies described herein can be produced by proteolytic cleavage of immunoglobulin molecules, using enzymes such as papain (to produce Fab fragments) or pepsin (to produce F(ab′)₂ fragments). F(ab′) 2 fragments contain the variable region, the light chain constant region and the C_{H}1 domain of the heavy chain. However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including the in vivo use of antibodies in humans as described herein and in vitro proliferation or cytotoxicity assays, it is preferable to use chimeric, humanized, or human antibodies.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

In some embodiments of the compositions, methods, and uses described herein, the AFP-FcRn inhibitor is a small molecule inhibitor, agent, or compound. In some embodiments of the aspects described herein, such AFP-FcRn small molecule inhibitors or small molecule inhibitors of AFP-FcRn can be used to inhibit or block the AFP binding site on FcRn, which overlaps with the albumin and IgG binding activities on FcRn, as described herein.

Such small molecule inhibitors include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

In some embodiments of these aspects and all such aspects described herein, the small molecule inhibitor of AFP-FcRn inhibits binding and/or interactions between Y521 and/or V522 of AFP and R42 of FcRn.

In some embodiments of these aspects and all such aspects described herein, the small molecule inhibitor of AFP-FcRn inhibits binding and/or interactions between P492 of AFP and R69 of FcRn.

In some embodiments of these aspects and all such aspects described herein, the small molecule inhibitor of AFP-FcRn inhibits binding and/or interactions between Q441 and/or V493 of AFP and E44 of FcRn.

In some embodiments of these aspects and all such aspects described herein, the small molecule inhibitor of AFP-FcRn inhibits binding and/or interactions between H534 and/or E589 of AFP and N173 of FcRn.

In some embodiments of these aspects and all such aspects described herein, the small molecule inhibitor of AFP-FcRn inhibits binding between the hydrophobic core of AFP and FcRn. In some such embodiments, the small molecule inhibitor of AFP-FcRn inhibits binding and/or interactions between L484, V493, V497, and/or F512 of AFP and V57, W59, and/or W61 of FcRn.

In some embodiments of these aspects and all such aspects described herein, the small molecule inhibitor of AFP-FcRn inhibits binding and/or interactions between T443 of AFP and E62 and/or W59 of FcRn.

In some embodiments of these aspects and all such aspects described herein, the small molecule inhibitor of AFP-FcRn inhibits binding and/or interactions between D529 of AFP and S230 of FcRn.

In some embodiments of these aspects and all such aspects described herein, the small molecule inhibitor of AFP-FcRn inhibits binding between S527 and/or D528 of AFP and E50 and/or 67Y of β2m complexed with FcRn.

In some embodiments of these aspects and all such aspects described herein, the small molecule inhibitor of AFP-FcRn inhibits binding between R604 of AFP and the carbonyl oxygen at E50 of β2m complexed with FcRn.

In some embodiments of these aspects and all such aspects described herein, the small molecule inhibitor of AFP-FcRn inhibits binding and/or interactions between Q597 of AFP and E69 of β2m complexed with FcRn.

In some embodiments of these aspects and all such aspects described herein the small molecule inhibitor of AFP-FcRn inhibits binding and/or interactions between E106 of AFP and H161 of FcRn.

In some embodiments of these aspects and all such aspects described herein, the small molecule inhibitor of AFP-FcRn inhibits binding and/or interactions between S135 of AFP and H161 of FcRn.

In some embodiments of these aspects and all such aspects described herein, the small molecule inhibitor of AFP-FcRn inhibits binding and/or interactions between F531, F533, F552, and/or F575 of AFP and W53 of FcRn.

In some embodiments of the compositions, methods, and uses described herein, an AFP-FcRn inhibitor is an RNA or DNA aptamer that binds or physically interacts with AFP, and blocks interactions between AFP and FcRn. In some embodiments of the compositions, methods, and uses described herein, an AFP-FcRn inhibitor is an RNA or DNA aptamer that binds or physically interacts with FcRn, and blocks interactions between AFP and FcRn. In some embodiments of the cocompositions, methods, and uses described herein, the aptamer comprises at least one RNA or DNA aptamer that binds to the FcRn large subunit p51 heavy chain precursor of FcRn. In some embodiments of the compositions, methods, and uses described herein, the aptamer comprises at least one RNA or DNA aptamer that binds to the β2m subunit of FcRn.

In some embodiments of these aspects and all such aspects described herein, the RNA or DNA aptamer inhibitor of AFP-FcRn inhibits binding and/or interactions between Y521 and/or V522 of AFP and R42 of FcRn.

In some embodiments of these aspects and all such aspects described herein, the RNA or DNA aptamer inhibitor of AFP-FcRn inhibits binding and/or interactions between P492 of AFP and R69 of FcRn.

In some embodiments of these aspects and all such aspects described herein, the RNA or DNA aptamer inhibitor of AFP-FcRn inhibits binding and/or interactions between Q441 and/or V493 of AFP and E44 of FcRn.

In some embodiments of these aspects and all such aspects described herein, the RNA or DNA aptamer inhibitor of AFP-FcRn inhibits binding and/or interactions between H534 and/or E589 of AFP and N173 of FcRn.

In some embodiments of these aspects and all such aspects described herein, the RNA or DNA aptamer inhibitor of AFP-FcRn inhibits binding between the hydrophobic core of AFP and FcRn. In some such embodiments, the RNA or DNA aptamer inhibitor of AFP-FcRn inhibits binding and/or interactions between L484, V493, V497, and/or F512 of AFP and V57, W59, and/or W61 of FcRn.

In some embodiments of these aspects and all such aspects described herein, the RNA or DNA aptamer inhibitor of AFP-FcRn inhibits binding and/or interactions between T443 of AFP and E62 and/or W59 of FcRn.

In some embodiments of these aspects and all such aspects described herein, the RNA or DNA aptamer inhibitor of AFP-FcRn inhibits binding and/or interactions between D529 of AFP and S230 of FcRn.

In some embodiments of these aspects and all such aspects described herein, the RNA or DNA aptamer inhibitor of AFP-FcRn inhibits binding between S527 and/or D528 of AFP and E50 and/or 67Y of β2m complexed with FcRn.

In some embodiments of these aspects and all such aspects described herein, the RNA or DNA aptamer inhibitor of AFP-FcRn inhibits binding between R604 of AFP and the carbonyl oxygen at E50 of β2m complexed with FcRn.

In some embodiments of these aspects and all such aspects described herein, the RNA or DNA aptamer inhibitor of AFP-FcRn inhibits binding and/or interactions between Q597 of AFP and E69 of β2m complexed with FcRn.

In some embodiments of these aspects and all such aspects described herein the RNA or DNA aptamer inhibitor of AFP-FcRn inhibits binding and/or interactions between E106 of AFP and H161 of FcRn.

In some embodiments of these aspects and all such aspects described herein, the RNA or DNA aptamer inhibitor of AFP-FcRn inhibits binding and/or interactions between S135 of AFP and H161 of FcRn.

In some embodiments of these aspects and all such aspects described herein, the RNA or DNA aptamer inhibitor of AFP-FcRn inhibits binding and/or interactions between F531, F533, F552, and/or F575 of AFP and W53 of FcRn.

AFP-FcRn inhibitors for use in the compositions, methods, and uses described herein can be identified or characterized using methods known in the art, such as protein-protein binding assays, biochemical screening assays, immunoassays, and cell-based assays, which are well known in the art, including, but not limited to, those described herein in the Examples and Figures.

For example, to identify a molecule that inhibits interaction between AFP and FcRn, transcytosis assays can be used, as described herein. For example, cells, such as MDCK II cells, that co-express human FcRn and β2m, can be tested for basolateral to apical transcytosis of AFP in the presence of a putative AFP-FcRn inhibitor (i.e., a test agent) and a control agent. If AFP transcytosis is inhibited by the presence of the test agent, relative to the transcytosis in the presence of the control agent, the test agent can be deemed a candidate inhibitor that inhibits binding between AFP and FcRn. Additional assays, such as, for example, Biacore assays, can be used to further determine whether and how a candidate agent inhibits binding between FcRn and AFP.

Also provided herein, in some aspects, are compositions, such as pharmaceutical compositions, comprising potentiators of AFP-FcRn interactions. Such potentiators are used to enhance/increase/potentiate the interaction between AFP and FcRn and/or increase transcytosis of human AFP, and/or increase serum half-life of AFP, thereby increasing immunosuppressive activities of AFP in the treatment of disorders and conditions in need of enhanced AFP levels, including autoimmune disorders, transplant patients, and high-risk pregnancies, for example.

As used herein, the terms “AFP-FcRn potentiator,” “potentiator of AFP-FcRn interaction, “AFP-FcRn activator agent,” and “AFP-FcRn agonist agent” refer to a molecule or agent that mimics or up-regulates (e.g., increases, potentiates or supplements) the biological activity of AFP binding to FcRn in vitro, in situ, and/or in vivo, including downstream pathways mediated by AFP binding to FcRn and signaling, such as, for example, transcytosis of AFP, inhibition of T cell stimulation by immune complex-primed dendritic cells, AFP-mediated inhibition of immune responses, and/or increased serum half-life of AFP. An AFP-FcRn potentiator or agonist can be, in some embodiments, an AFP protein fragment or derivative thereof having at least one bioactivity of the wild-type AFP. An AFP-FcRn potentiator can also be a compound which increases the interaction of AFP with FcRn, for example. Exemplary AFP-FcRn potentiators or agonists contemplated for use in the various aspects and embodiments described herein include, but are not limited to, antibodies or antigen-binding fragments thereof that specifically bind to AFP bound to FcRn and enhance the interaction and/or block FcRn binding to albumin and/or IgG but allow binding of AFP to FcRn; RNA or DNA aptamers that bind to FcRn and mimic AFP binding to FcRn; AFP structural analogs or AFP functional fragments, derivatives, or fusion polypeptides thereof; and small molecule agents that target or bind to FcRn and act as functional mimics of AFP binding to FcRn.

As used herein, an AFP-FcRn potentiator has the ability to increase or enhance the activity of AFP binding to FcRn or mimic/replicate the downstream functional consequences mediated by AFP binding to FcRn by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the AFP-FcRn potentiator.

In some embodiments of these aspects and all such aspects described herein, an AFP-FcRn potentiator comprises a G109R, R487S, and/or S445L polymorphism of wild-type AFP that increases AFP-FcRn binding.

In some embodiments of these aspects and all such aspects described herein, an AFP-FcRn potentiator enhances binding and/or interactions between Y521 and/or V522 of AFP and R42 of FcRn.

In some embodiments of these aspects and all such aspects described herein, an AFP-FcRn potentiator enhances binding and/or interactions between P492 of AFP and R69 of FcRn.

In some embodiments of these aspects and all such aspects described herein, an AFP-FcRn potentiator enhances binding and/or interactions between Q441 and/or V493 of AFP and E44 of FcRn.

In some embodiments of these aspects and all such aspects described herein, an AFP-FcRn potentiator enhances binding and/or interactions between H534 and/or E589 of AFP and N173 of FcRn.

In some embodiments of these aspects and all such aspects described herein, an AFP-FcRn potentiator enhances binding between the hydrophobic core of AFP and FcRn. In some such embodiments, an AFP-FcRn potentiator enhances binding and/or interactions between L484, V493, V497, and/or F512 of AFP and V57, W59, and/or W61 of FcRn.

In some embodiments of these aspects and all such aspects described herein, an AFP-FcRn potentiator enhances binding and/or interactions between T443 of AFP and E62 and/or W59 of FcRn.

In some embodiments of these aspects and all such aspects described herein, an AFP-FcRn potentiator enhances binding and/or interactions between D529 of AFP and S230 of FcRn.

In some embodiments of these aspects and all such aspects described herein, an AFP-FcRn potentiator enhances binding between S527 and/or D528 of AFP and E50 and/or 67Y of β2m complexed with FcRn.

In some embodiments of these aspects and all such aspects described herein, an AFP-FcRn potentiator enhances binding between R604 of AFP and the carbonyl oxygen at E50 of β2m complexed with FcRn.

In some embodiments of these aspects and all such aspects described herein, an AFP-FcRn potentiator enhances binding and/or interactions between Q597 of AFP and E69 of β2m complexed with FcRn.

In some embodiments of these aspects and all such aspects described herein, an AFP-FcRn potentiator enhances binding and/or interactions between E106 of AFP and H161 of FcRn.

In some embodiments of these aspects and all such aspects described herein, an AFP-FcRn potentiator enhances binding and/or interactions between S135 of AFP and H161 of FcRn.

In some embodiments of these aspects and all such aspects described herein, an AFP-FcRn potentiator enhances binding and/or interactions between F531, F533, F552, and/or F575 of AFP and W53 of FcRn.

In some embodiments of the compositions, methods, and uses described herein, the AFP-FcRn potentiator is an antibody or antigen-binding fragment thereof that selectively binds or physically interacts with AFP bound to FcRn and enhances the interaction of AFP and FcRn, and/or blocks FcRn binding to albumin and/or IgG but allows binding of AFP to FcRn, thereby resulting in increased transcytosis of AFP, increased inhibition of T cell stimulation by immune complex-primed dendritic cells, increased AFP-mediated inhibition of immune responses, and/or increased serum half-life of AFP. Exemplary assays to measure increases or up-regulation of downstream pathway activities are known to those of ordinary skill in the art and are provided herein in the Examples.

In some embodiments of the compositions, methods, and uses described herein, an AFP-FcRn potentiator is a monoclonal antibody. In some embodiments of the compositions, methods, and uses described herein, an AFP-FcRn potentiator is an antibody fragment or antigen-binding fragment, as the term is described elsewhere herein.

In some embodiments of the compositions, methods, and uses described herein, an AFP-FcRn potentiator is a chimeric antibody derivative of the AFP-FcRn potentiator antibodies and antigen-binding fragments thereof, as the term is described elsewhere herein.

In some embodiments of the compositions, methods, and uses described herein, an AFP-FcRn potentiator is a humanized antibody derivative, as the term is described elsewhere herein.

In some embodiments, the AFP-FcRn potentiator antibodies and antigen-binding fragments thereof described herein include derivatives that are modified, i.e., by the covalent attachment of any type of molecule to the antibody, provided that covalent attachment does not prevent the antibody from binding to the target antigen.

In some embodiments, the AFP-FcRn potentiator antibodies and antigen-binding fragments thereof described herein are completely human antibodies or antigen-binding fragments thereof, which are particularly desirable for the therapeutic treatment of human patients. Human antibodies can be made by a variety of methods known in the art, and as described in more detail elsewhere herein.

The AFP-FcRn potentiator antibodies and antigen-binding fragments thereof described herein, as well as any of the other antibodies or antigen-binding fragments thereof described herein in various aspects and embodiments, can be generated by any suitable method known in the art.

In some embodiments of the compositions, methods, and uses described herein, the AFP-FcRn potentiator is a small molecule potentiator, activator, or agonist, including, but not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, about 1000 to about 10,000 Da. In some embodiments of the compositions, methods, and uses described herein, an AFP-FcRn potentiator comprises a small molecule that binds FcRn and mimics AFP binding. Exemplary sites of small molecule binding include, but are not limited to, the portion of FcRn that binds specifically to AFP, or to the portions of FcRn adjacent to the AFP binding site.

In some embodiments of the compositions, methods, and uses described herein, an AFP-FcRn potentiator is an RNA or DNA aptamer that binds or physically interacts with AFP or FcRn, and enhances or promotes interactions between AFP and FcRn.

In some embodiments of the compositions, methods, and uses described herein, an AFP-FcRn potentiator comprises an AFP structural analog, functional fragment, or derivative, such as an AFP variant engineered to possess increased binding to the FcRn/β2-microglobulin complex. The term “AFP structural analog,” “AFP functional fragment,” or “AFP derivative” as used herein, refer to compounds, such as peptides, that can bind to FcRn under physiological conditions in vitro or in vivo, wherein the binding at least partially mimics or increases an FcRn mediated biological activity. Suitable AFP structural analogs, functional fragments, or derivatives can be designed and synthesized through molecular modeling of AFP binding to FcRn, for example.

In some embodiments of these aspects and all such aspects described herein, an AFP-functional fragment comprises Y521 and/or V522 of AFP and can interact with R42 of FcRn.

In some embodiments of these aspects and all such aspects described herein, an AFP-functional fragment comprises P492 of AFP and can interact with R69 of FcRn.

In some embodiments of these aspects and all such aspects described herein, an AFP-functional fragment comprises Q441 and/or V493 of AFP and can interact with E44 of FcRn.

In some embodiments of these aspects and all such aspects described herein, an AFP-functional fragment comprises H534 and/or E589 of AFP and can interact with N173 of FcRn.

In some embodiments of these aspects and all such aspects described herein, an AFP-functional fragment comprises L484, V493, V497, and/or F512 of AFP and can interact with V57, W59, and/or W61 of FcRn.

In some embodiments of these aspects and all such aspects described herein, an AFP-functional fragment comprises T443 of AFP and can interact with E62 and/or W59 of FcRn.

In some embodiments of these aspects and all such aspects described herein, an AFP-functional fragment comprises D529 of AFP and can interact with S230 of FcRn.

In some embodiments of these aspects and all such aspects described herein, an AFP-functional fragment comprises S527 and/or D528 of AFP and can interact with E50 and/or 67Y of β2m complexed with FcRn.

In some embodiments of these aspects and all such aspects described herein, an AFP-functional fragment comprises R604 of AFP and can interact with the carbonyl oxygen at E50 of β2m complexed with FcRn.

In some embodiments of these aspects and all such aspects described herein, an AFP-functional fragment comprises Q597 of AFP and can interact with E69 of β2m complexed with FcRn.

In some embodiments of these aspects and all such aspects described herein, an AFP-functional fragment comprises E106 of AFP and can interact with H161 of FcRn.

In some embodiments of these aspects and all such aspects described herein, an AFP-functional fragment comprises S135 of AFP and can interact with H161 of FcRn.

In some embodiments of these aspects and all such aspects described herein, an AFP-functional fragment comprises F531, F533, F552, and/or F575 of AFP and can interact with W53 of FcRn.

In some embodiments of these aspects and all such aspects described herein, an AFP-functional fragment comprises

(SEQ ID NO: 4)
MKWVESIFLIFLLNFTESRTLHRNEYGIASILDSYQCTAEISLADLATIF
FAQFVQEATYKEVSKMVKDALTAIEKPTGDEQSSGCLENQLPAFLEELCH
EKEILEKYGHSDCCSQSEEGRHNCFLAHKKPTPASIPLFQVPEPVTSCEA
YEEDRETFMNKFIYEIARRHPFLYAPTILLWAARYDKIIPSCCKAENAVE
CFQTKAATVTKELRESSLLNQHACAVMKNFGTRTFQAITVTKLSQKFTKV
NFTEIQKLVLDVAHVHEHCCRGDVLDCLQDGEKIMSYICSQQDTLSNKIT
ECCKLTTLERGQCIIHAENDEKPEGLSPNLNRFLGDRDFNQFSSGEKNIF
LASFVHEYSRRHPQLAVSVILRVAKGYQELLEKCFQTENPLECQDKGEEE
LQKYIQESQALAKRSCGLFQKLGEYYLQNAFLVAYTKKAPQLTSSELMAI
TRKMAATAATCCQLSEDKLLACGEGAADIIIGHLCIRHEMTPVNPGVGQC
CTSSYANRRPCFSSLVVDETYVPPAFSDDKFIFHKDLCQAQGVALQTMKQ
EFLINLVKQKPQITEEQLEAVIADF
or
AFP (1-575).

In some embodiments of these aspects and all such aspects described herein, an AFP-functional fragment comprises

(SEQ ID NO: 5)
LCIRHEMTPVNPGVGQCCTSSYANRRPCFSSLVVDETYVPPAFSDDKFIF
HKDLCQAQGVALQTMKQEFLINLVKQKPQITEEQLEAVIADF
or
AFP (484-575).

The term “AFP functional fragment,” as used herein, refers to a fragment of AFP that specifically binds to FcRn and can be transcytosed across a cell membrane, and can, in some embodiments, increase serum half-life of a protein to which it is fused or conjugated. Accordingly, the term “functional” when used in conjunction with a fragment, “derivative” or “variant” refers to a protein molecule that possesses a desired biological activity that is substantially similar to a biological activity of the entity or molecule of which it is a fragment, derivative or variant. By “substantially similar” in this context is meant that the biological activity, e.g., specifically bind to FcRn and be transcytosed across a cell membrane, and can, in some embodiments, increase serum half-life of a protein to which it is fused or conjugated, is at least 50% as active as a reference, e.g., a corresponding wild-type or endogenous AFP, and preferably at least 60% as active, 70% as active, 80% as active, 90% as active, 95% as active, 100% as active or even higher (i.e., the variant or derivative has greater activity than the wild-type), e.g., 110% as active, 120% as active, or more. Assays to measure the biological activity of an AFP functional fragment are known in the art, and non-limiting examples are provided herein in the Examples.

In some embodiments of these aspects and all such aspects described herein, an AFP-functional fragment differs from endogenous AFP by one or more amino acid or nucleic acid deletions, additions, substitutions or side-chain modifications, yet retains one or more functions or biological activities of the naturally occurring molecule. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide).

As used herein the term “derivative” refers to a polypeptide that is derived from wild-type AFP as described herein, e.g., an AFP functional fragment, and includes peptides which have been chemically modified by techniques such as adding additional side chains, ubiquitination, labeling, pegylation (derivatization with polyethylene glycol), and insertion, deletion or substitution of amino acid mimetics and/or unnatural amino acids that do not normally occur in the sequence of wild-type AFP that is basis of the derivative. For example, in some embodiments, an AFP derivative can comprise a label, such as, for example, an epitope, e.g., a FLAG epitope or a V5 epitope or an HA epitope. Such a tag can be useful for, for example, purifying a fusion protein derivative. The term “derivative” also encompasses a derivatized polypeptide, such as, for example, a polypeptide modified to contain one or more-chemical moieties other than an amino acid. The chemical moiety can be linked covalently to the peptide, e.g., via an amino terminal amino acid residue, a carboxy terminal amino acid residue, or at an internal amino acid residue. Such modifications include the addition of a protective or capping group on a reactive moiety in the polypeptide, addition of a detectable label, and other changes that do not adversely destroy the activity of the AFP derivative or fusion protein. In some embodiments, an AFP derivative contains additional chemical moieties not normally a part of the molecule. Such moieties can improve its solubility, absorption, biological half life, etc. The moieties can alternatively decrease the toxicity of the molecule, or eliminate or attenuate an undesirable side effect of the molecule, etc. Moieties capable of mediating such effects are disclosed, for example, in Remington's Pharmaceutical Sciences, 18th edition, A. R. Gennaro, Ed., MackPubl., Easton, PA (1990).

In some embodiments, an AFP functional fragment comprises an AFP functional fragment that differs by 1 conservative substitution, 2 conservative substitutions, 3 conservative substitutions, 4 conservative substitutions, 5 conservative substitutions, 6 conservative substitutions, 7 conservative substitutions, 8 conservative substitutions, 9 conservative substitutions, 10 or fewer conservative substitutions, 15 or fewer conservative substitutions, 20 or fewer conservative substitutions, 25 or fewer conservative substitutions, 30 or fewer conservative substitutions, 35 or fewer conservative substitutions, 40 or fewer conservative substitutions, 45 or fewer conservative substitutions, or 50 or fewer conservative substitutions, relative to the sequence of the naturally occurring AFP molecule or a domain or portion thereof of AFP having the desired biological activity.

In some embodiments, an AFP functional fragment differs by 1 or fewer non-conservative substitutions, 2 or fewer non-conservative substitutions, 3 or fewer non-conservative substitutions, 4 or fewer non-conservative substitutions, 5 or fewer non-conservative substitutions, 6 or fewer non-conservative substitutions, 7 or fewer or non-conservative substitutions, 8 or fewer non-conservative substitutions, 9 or fewer non-conservative substitutions, 10 or fewer or non-conservative substitutions, 15 or fewer or non-conservative substitutions, relative to the sequence of the naturally occurring AFP molecule or a domain or portion thereof of AFP having the desired biological activity.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

AFP-FcRn potentiators for use in the compositions, methods, and uses described herein can be identified or characterized using methods known in the art, such as protein-protein binding assays, biochemical screening assays, immunoassays, and cell-based assays, which are well known in the art, such as those described herein in the Examples.

For the clinical use of the methods and uses described herein, administration of the compositions comprising AFP-FcRn inhibitors or AFP-FcRn potentiators can include formulation into pharmaceutical compositions or pharmaceutical formulations for parenteral administration, e.g., intravenous; mucosal, e.g., intranasal; ocular, or other mode of administration. In some embodiments, the AFP-FcRn inhibitors or AFP-FcRn potentiators described herein, can be administered along with any pharmaceutically acceptable carrier compound, material, or composition which results in an effective treatment in the subject. Thus, a pharmaceutical formulation for use in the methods described herein can contain an AFP-FcRn inhibitors or AFP-FcRn potentiator as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of an AFP-FcRn inhibitor or AFP-FcRn potentiator. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The AFP-FcRn inhibitors or AFP-FcRn potentiators described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, an AFP-FcRn inhibitor or AFP-FcRn potentiator can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of the compositions comprising AFP-FcRn inhibitors or AFP-FcRn potentiators that can be used in the methods described herein are described below.

Parenteral Dosage Forms. Parenteral dosage forms of the AFP-FcRn inhibitors or AFP-FcRn potentiators can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. An AFP-FcRn inhibitor or AFP-FcRn potentiator can be packaged in a pressurized aerosol container together with suitable propellants, for example, hydrocarbon propellants like propane, butane, or isobutane with conventional adjuvants. An AFP-FcRn inhibitor or AFP-FcRn potentiator described herein, can also be administered in a non-pressurized form such as in a nebulizer or atomizer. An AFP-FcRn inhibitor or AFP-FcRn potentiator described herein, can also be administered directly to the airways in the form of a dry powder, for example, by use of an inhaler.

Suitable powder compositions include, by way of illustration, powdered preparations of AFP-FcRn inhibitors or AFP-FcRn potentiators described herein, thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the AFP-FcRn inhibitors or AFP-FcRn potentiators described herein, further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations overtime. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the aspects described herein, AFP-FcRn inhibitors or AFP-FcRn potentiators can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Chemg-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an AFP-FcRn inhibitor or AFP-FcRn potentiator is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the AFP-FcRn inhibitors or AFP-FcRn potentiators described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments of the methods described herein, the AFP-FcRn inhibitors or AFP-FcRn potentiators for use in the methods described herein are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the AFP-FcRn inhibitors or AFP-FcRn potentiators described herein administered over the course of treatment to the subject or patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Methods of Treatment and Uses of AFP-FcRn Inhibitors and Potentiators

As demonstrated herein, alpha-fetoprotein (AFP) is a third ligand for the neonatal Fc receptor and soluble human FcRn binds to AFP with affinities greater than observed with albumin, and less than that of IgG. As further shown herein, the AFP binding site on FcRn interferes with the albumin and IgG binding activities of FcRn, and antibodies that are specific for the albumin site on hFcRn can decrease FcRn-mediated AFP transport. As also demonstrated herein, the binding of FcRn to AFP occurs over a much wider pH range than that observed for IgG and albumin, which typically bind under acidic pH conditions. In addition, provided herein are single nucleotide polymorphisms in AFP that can impact binding of AFP with human FcRn, such as, for example, G109R, R487S, and S445L that increase AFP-FcRn binding, and T451I and D536V, that decrease AFP-FcRn binding.

Accordingly, provided herein, in some aspects, are methods to inhibit or reduce FcRn and AFP interactions in diseases or disorders where elevated AFP levels are associated with immunosuppression comprising administering a therapeutically effective amount of a pharmaceutical composition comprising an AFP-FcRn inhibitor to a subject in need thereof.

In some embodiments of these aspects and all such aspects described herein, an AFP-FcRn inhibitor comprises a T451I and/or D536V polymorphism of wild-type AFP that decreases AFP-FcRn binding.

In some embodiments of these aspects and all such aspects described herein, an AFP-FcRn inhibitor inhibits binding and/or interactions between Y521 and/or V522 of AFP and R42 of FcRn.

In some embodiments of these aspects and all such aspects described herein, an AFP-FcRn inhibitor inhibits binding and/or interactions between P492 of AFP and R69 of FcRn.

In some embodiments of these aspects and all such aspects described herein, an AFP-FcRn inhibitor inhibits binding and/or interactions between Q441 and/or V493 of AFP and E44 of FcRn.

In some embodiments of these aspects and all such aspects described herein, an AFP-FcRn inhibitor inhibits binding and/or interactions between H534 and/or E589 of AFP and N173 of FcRn.

In some embodiments of these aspects and all such aspects described herein, an AFP-FcRn inhibitor inhibits binding between the hydrophobic core of AFP and FcRn. In some such embodiments, an AFP-FcRn inhibitor inhibits binding and/or interactions between L484, V493, V497, and/or F512 of AFP and V57, W59, and/or W61 of FcRn.

In some embodiments of these aspects and all such aspects described herein, an AFP-FcRn inhibitor inhibits binding and/or interactions between T443 of AFP and E62 and/or W59 of FcRn.

In some embodiments of these aspects and all such aspects described herein, an AFP-FcRn inhibitor inhibits binding and/or interactions between D529 of AFP and S230 of FcRn.

In some embodiments of these aspects and all such aspects described herein, an AFP-FcRn inhibitor inhibits binding between S527 and/or D528 of AFP and E50 and/or 67Y of β2m complexed with FcRn.

In some embodiments of these aspects and all such aspects described herein, an AFP-FcRn inhibitor inhibits binding between R604 of AFP and the carbonyl oxygen at E50 of β2m complexed with FcRn.

In some embodiments of these aspects and all such aspects described herein, an AFP-FcRn inhibitor inhibits binding and/or interactions between Q597 of AFP and E69 of β2m complexed with FcRn.

In some embodiments of these aspects and all such aspects described herein, an AFP-FcRn inhibitor inhibits binding and/or interactions between E106 of AFP and H161 of FcRn.

In some embodiments of these aspects and all such aspects described herein, an AFP-FcRn inhibitor inhibits binding and/or interactions between S135 of AFP and H161 of FcRn.

In some embodiments of these aspects and all such aspects described herein, an AFP-FcRn inhibitor inhibits binding and/or interactions between F531, F533, F552, and/or F575 of AFP and W53 of FcRn.

In some embodiments of these aspects and all such aspects described herein, a subject having a disease or disorder associated with elevated AFP levels has or has been diagnosed with cancer.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In some embodiments of these methods and all such methods described herein, a subject having a disease or disorder associated with elevated AFP levels has or has been diagnosed with a cancer or tumor of primitive origin, a tumor of liver origin, such as a hepatoma, a tumor of biliary origin, such as cholangiocarcinoma, stomach cancer, pancreatic cancer, or a teratocarcinoma.

In some embodiments of these methods and all such methods described herein, the methods further comprise administering an anti-cancer therapy or agent to a subject in addition to the AFP-FcRn inhibitors described herein.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets PD1, PDL1, PDL2, TIM3 or any TIM family member, CEACAM1 or any CEACAM family member, ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated for the methods described herein.

In some embodiments, an anti-cancer therapy comprises an immunotherapy such as adoptive cell transfer. “Adoptive cell transfer,” as used herein, includes immunotherapies involving genetically engineering a subject or patient's own T cells to produce special receptors on their surface called chimeric antigen receptors (CARs). CARs are proteins that allow the T cells to recognize a specific protein (antigen) on tumor cells. These engineered CAR T cells are then grown in the laboratory until they number in the billions. The expanded population of CAR T cells is then infused into the patient. After the infusion, the T cells multiply in the subject's body and, with guidance from their engineered receptor, recognize and kill cancer cells that harbor the antigen on their surfaces.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including active fragments and/or variants thereof.

In some embodiments of these methods and all such methods described herein, the methods further comprise administering a chemotherapeutic agent to the subject being administered the AFP-FcRn inhibitors described herein.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

In some embodiments of these methods and all such methods described herein, the methods further comprise administering a tumor or cancer antigen to a subject being administered the AFP-FcRn inhibitors described herein.

A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

By “reduce” or “inhibit” in terms of the cancer treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. Reduce or inhibit can refer to, for example, the symptoms of the disorder being treated, the presence or size of metastases or micrometastases, the size of the primary tumor, the presence or the size of the dormant tumor, etc.

As used herein, “alleviating a symptom of a cancer or tumor” is ameliorating any condition or symptom associated with the cancer such as the symptoms of the cancer being treated, the presence or size of metastases or micrometastases, the size of the primary tumor, the presence or the size of the dormant tumor, etc. As compared with an equivalent untreated control, such as a subject prior to the administration of the AFP-FcRn inhibitors, such reduction or degree of prevention is at least 5%, 10%, 20%, 40%, 50%, 60%, 80%, 90%, 95%, or more as measured by any standard technique known to one of ordinary skill in the art. A patient or subject who is being treated for a cancer or tumor is one who a medical practitioner has diagnosed as having such a condition. Diagnosis can be by any suitable means.

Also provided herein, in some aspects, are methods to increase or potentiate FcRn and AFP interactions in diseases or disorders associated with decreased AFP levels or where increasing AFP levels is beneficial comprising administering a therapeutically effective amount of a pharmaceutical composition comprising an AFP-FcRn potentiator to a subject in need thereof.

In some embodiments of these aspects and all such aspects described herein, an AFP-FcRn potentiator comprises a G109R, R487S, and/or S445L polymorphism of wild-type AFP that increases AFP-FcRn binding.

In some embodiments of these aspects and all such aspects described herein, an AFP-FcRn potentiator enhances binding and/or interactions between Y521 and/or V522 of AFP and R42 of FcRn.

In some embodiments of these aspects and all such aspects described herein, an AFP-FcRn potentiator enhances binding and/or interactions between P492 of AFP and R69 of FcRn.

In some embodiments of these aspects and all such aspects described herein, an AFP-FcRn potentiator enhances binding and/or interactions between Q441 and/or V493 of AFP and E44 of FcRn.

In some embodiments of these aspects and all such aspects described herein, an AFP-FcRn potentiator enhances binding and/or interactions between H534 and/or E589 of AFP and N173 of FcRn.

In some embodiments of these aspects and all such aspects described herein, an AFP-FcRn potentiator enhances binding between the hydrophobic core of AFP and FcRn. In some such embodiments, an AFP-FcRn potentiator enhances binding and/or interactions between L484, V493, V497, and/or F512 of AFP and V57, W59, and/or W61 of FcRn.

In some embodiments of these aspects and all such aspects described herein, an AFP-FcRn potentiator enhances binding and/or interactions between T443 of AFP and E62 and/or W59 of FcRn.

In some embodiments of these aspects and all such aspects described herein, an AFP-FcRn potentiator enhances binding and/or interactions between D529 of AFP and S230 of FcRn.

In some embodiments of these aspects and all such aspects described herein, an AFP-FcRn potentiator enhances binding between S527 and/or D528 of AFP and E50 and/or 67Y of β2m complexed with FcRn.

In some embodiments of these aspects and all such aspects described herein, an AFP-FcRn potentiator enhances binding between R604 of AFP and the carbonyl oxygen at E50 of β2m complexed with FcRn.

In some embodiments of these aspects and all such aspects described herein, an AFP-FcRn potentiator enhances binding and/or interactions between Q597 of AFP and E69 of β2m complexed with FcRn.

In some embodiments of these aspects and all such aspects described herein, an inhibitor of AFP-FcRn inhibits binding and/or interactions between E106 of AFP and H161 of FcRn.

In some embodiments of these aspects and all such aspects described herein, an inhibitor of AFP-FcRn inhibits binding and/or interactions between S135 of AFP and H161 of FcRn.

In some embodiments of these aspects and all such aspects described herein, an inhibitor of AFP-FcRn inhibits binding and/or interactions between F531, F533, F552, and/or F575 of AFP and W53 of FcRn.

In some embodiments of the compositions, methods, and uses described herein, the AFP-FcRn potentiator is an antibody or antigen-binding fragment thereof that selectively binds or physically interacts with AFP bound to FcRn and enhances the interaction of AFP and FcRn, and/or blocks FcRn binding to albumin and/or IgG but allows binding of AFP to FcRn, thereby resulting in increased transcytosis of AFP, increased inhibition of T cell stimulation by immune complex-primed dendritic cells, increased AFP-mediated inhibition of immune responses, and/or increased serum half-life of AFP.

In some embodiments of these methods and all such methods described herein, a subject in need of increased AFP levels or increased AFP and FcRn interaction or binding is a pregnant subject.

In some embodiments of these methods and all such methods described herein, a subject in need of increased AFP levels or increased AFP and FcRn interaction or binding is a subject at risk for having a problem with establishing and/or maintaining a pregnancy.

In some embodiments, prior to administrating the pharmaceutical compositions comprising AFP-FcRn potentiators described herein to a subject, a subject is first identified as one who has one or more of the following: placental insufficiency (See, e.g., Lepercq and Mahieu-Caputo, 1998, Horm. Res. 49(suppl 2):14-19); a specific maternal weight and/or height prior to pregnancy (e.g., that is identified as being at risk for non full-term pregnancy); low weight gain during pregnancy; maternal history of non-full term pregnancies (e.g., spontaneous abortion, stillbirth, neonatal death, and/or premature parturition); previous offspring with low birth weight; specific maternal activities placing the pregnancy at risk (e.g., smoking, alcohol and/or drug use, and/or poor nutrition); early intrauterine infections; maternal medical diseases; multiparous pregnancies; a history of or newly experienced complications arising during pregnancy; and/or a general desire for establishing pregnancy and/or maintenance of a healthy pregnancy.

In some embodiments of these methods and all such methods described herein, a subject in need of increased AFP levels or increased AFP and FcRn interaction or binding has or has been diagnosed with an autoimmune disease or disorder.

The methods described herein can, in some aspects, be employed in the context of treatment for an autoimmune disease.

Accordingly, in some embodiments of these methods and all such methods described herein, the autoimmune diseases to be treated or prevented using the methods described herein, include, but are not limited to: rheumatoid arthritis, Crohn's disease or colitis, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Autoimmune disease has been recognized also to encompass atherosclerosis and Alzheimer's disease. In some embodiments of the aspects described herein, the autoimmune disease is selected from the group consisting of multiple sclerosis, type-I diabetes, Hashimoto's thyroiditis, Crohn's disease or colitis, rheumatoid arthritis, systemic lupus erythematosus, gastritis, autoimmune hepatitis, hemolytic anemia, autoimmune hemophilia, autoimmune lymphoproliferative syndrome (ALPS), autoimmune uveoretinitis, glomerulonephritis, Guillain-Barre syndrome, psoriasis and myasthenia gravis.

In some embodiments of these methods and all such methods described herein, a subject in need of increased AFP levels or increased AFP and FcRn interaction or binding has or has been diagnosed with host versus graft disease (HVGD). In a further such embodiment, the subject being treated with the methods described herein is an organ or tissue transplant recipient. In some embodiments, the methods are used for increasing transplantation tolerance in a subject. In some such embodiments, the subject is a recipient of an allogenic transplant.

The transplant can be any organ or tissue transplant, including but not limited to heart, kidney, liver, skin, pancreas, bone marrow, skin or cartilage. “Transplantation tolerance,” as used herein, refers to a lack of rejection of the donor organ by the recipient's immune system.

As used herein, in regard to any of the compositions, methods, and uses comprising AFP-FcRn inhibitors or potentiators described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The terms “subject,” “patient,” and “individual” as used in regard to any of the methods described herein are used interchangeably herein, and refer to an animal, for example a human, recipient of the inhibitors described herein. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like. Production mammal, e.g. cow, sheep, pig, and the like are also encompassed in the term subject.

The term “effective amount” as used herein refers to the amount of a AFP-FcRn inhibitor or potentiator described herein, needed to alleviate at least one or more symptom of the disease or disorder being treated, and relates to a sufficient amount of pharmacological composition to provide the desired effect, e.g., increase or decrease serum AFP levels. The term “therapeutically effective amount” therefore refers to an amount of the inhibitors or potentiators described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The AFP-FcRn inhibitors or potentiators described herein can be administered to a subject in need thereof by any appropriate route which results in an effective treatment in the subject. As used herein, the terms “administering,” and “introducing” are used interchangeably and refer to the placement of an AFP-FcRn inhibitors or potentiators into a subject by a method or route which results in at least partial localization of such agents at a desired site, such as a tumor site or site of inflammation, such that a desired effect(s) is produced.

In some embodiments, the AFP-FcRn inhibitors or potentiators described herein can be administered to a subject by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the AFP-FcRn inhibitors or potentiators, other than directly into a target site, tissue, or organ, such that it enters the subject's circulatory system and, thus, is subject to metabolism and other like processes.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Exemplary embodiments of the various aspects disclosed herein can be described by one or more of the following numbered paragraphs:
A. A pharmaceutical composition comprising an inhibitor of AFP-FcRn and a pharmaceutically acceptable carrier, wherein said inhibitor of AFP-FcRn inhibits binding between alpha-fetoprotein (AFP) and FcRn.
B. The pharmaceutical composition of paragraph A, wherein the inhibitor of AFP-FcRn comprises a T451I and/or D536V polymorphism of wild-type AFP.
C. The pharmaceutical composition of any one of paragraphs A-B, wherein the inhibitor of AFP-FcRn inhibits binding between Y521 and/or V522 of AFP and R42 of FcRn.
D. The pharmaceutical composition of any one of paragraphs A-C, wherein the inhibitor of AFP-FcRn inhibits binding between P492 of AFP and R69 of FcRn.
E. The pharmaceutical composition of any one of paragraphs A-D, wherein the inhibitor of AFP-FcRn inhibits binding between Q441 and/or V493 of AFP and E44 of FcRn.
F. The pharmaceutical composition of any one of paragraphs A-E, wherein the inhibitor of AFP-FcRn inhibits binding between H534 and/or E589 of AFP and N173 of FcRn.
G. The pharmaceutical composition of any one of paragraphs A-F, wherein the inhibitor of AFP-FcRn inhibits binding between the hydrophobic core of AFP and FcRn.
H. The pharmaceutical composition of any one of paragraphs A-G, wherein the inhibitor of AFP-FcRn inhibits binding between L484, V493, V497, and/or F512 of AFP and V57, W59, and/or W61 of FcRn.
I. The pharmaceutical composition of any one of paragraphs A-H, wherein the inhibitor of AFP-FcRn inhibits binding between T443 of AFP and E62 and/or W59 of FcRn.
J. The pharmaceutical composition of any one of paragraphs A-I, wherein the inhibitor of AFP-FcRn inhibits binding between D529 of AFP and S230 of FcRn.
K. The pharmaceutical composition of any one of paragraphs A-J, wherein the inhibitor of AFP-FcRn inhibits binding between S527 and/or D528 of AFP and E50 and/or 67Y of β2m complexed with FcRn.
L. The pharmaceutical composition of any one of paragraphs A-K, wherein the inhibitor of AFP-FcRn inhibits binding between R604 of AFP and the carbonyl oxygen at E50 of β2m complexed with FcRn.
M. The pharmaceutical composition of any one of paragraphs A-L, wherein the inhibitor of AFP-FcRn inhibits binding between Q597 of AFP and E69 of β2m complexed with FcRn.
N. The pharmaceutical composition of any one of paragraphs A-M, wherein the inhibitor of AFP-FcRn inhibits binding between E106 of AFP and H161 of FcRn.
O. The pharmaceutical composition of any one of paragraphs A-N, wherein the inhibitor of AFP-FcRn inhibits binding between S135 of AFP and H161 of FcRn.
P. The pharmaceutical composition of any one of paragraphs A-O, wherein the inhibitor of AFP-FcRn inhibits binding between F531, F533, F552, and/or F575 of AFP and W53 of FcRn.
Q. The pharmaceutical composition of any one of paragraphs A-P, wherein the inhibitor of AFP-FcRn is an antibody or antigen-binding fragment thereof, a small molecule compound, or an RNA or DNA aptamer.
R. The pharmaceutical composition of paragraph Q, wherein the antibody or antigen-binding fragment thereof is a chimeric, humanized, or completely human antibody or antigen-binding fragment thereof.
S. The pharmaceutical composition of any one of paragraphs A-R, wherein the inhibitor of AFP-FcRn inhibits or blocks the AFP binding site on FcRn.
T. A pharmaceutical composition comprising an AFP-FcRn potentiator and a pharmaceutically acceptable carrier.
U. The pharmaceutical composition of paragraph T, wherein the AFP-FcRn potentiator comprises a G109R, R487S, and/or S445L polymorphism of wild-type alpha-fetoprotein (AFP) that increases AFP-FcRn binding.
V. The pharmaceutical composition of any one of paragraphs T-U, wherein the AFP-FcRn potentiator enhances binding between Y521 and/or V522 of AFP and R42 of FcRn.
W. The pharmaceutical composition of any one of paragraphs T-V, wherein the AFP-FcRn potentiator enhances binding between P492 of AFP and R69 of FcRn.
X. The pharmaceutical composition of any one of paragraphs T-W, wherein the AFP-FcRn potentiator enhances binding between Q441 and/or V493 of AFP and E44 of FcRn.
Y. The pharmaceutical composition of any one of paragraphs T-X, wherein the AFP-FcRn potentiator enhances binding between H534 and/or E589 of AFP and N173 of FcRn.
Z. The pharmaceutical composition of any one of paragraphs T-Y, wherein the AFP-FcRn potentiator enhances binding between the hydrophobic core of AFP and FcRn.
AA. The pharmaceutical composition of any one of paragraphs T-Z, wherein the AFP-FcRn potentiator enhances binding between L484, V493, V497, and/or F512 of AFP and V57, W59, and/or W61 of FcRn.
BB. The pharmaceutical composition of any one of paragraphs T-AA, wherein the AFP-FcRn potentiator enhances binding between T443 of AFP and E62 and/or W59 of FcRn.
CC. The pharmaceutical composition of any one of paragraphs T-BB, wherein the AFP-FcRn potentiator enhances binding between D529 of AFP and S230 of FcRn.
DD. The pharmaceutical composition of any one of paragraphs T-CC, wherein the AFP-FcRn potentiator enhances binding between S527 and/or D528 of AFP and E50 and/or 67Y of β2m complexed with FcRn.
EE. The pharmaceutical composition of any one of paragraphs T-DD, wherein the AFP-FcRn potentiator enhances binding between R604 of AFP and the carbonyl oxygen at E50 β2m complexed with FcRn.
FF. The pharmaceutical composition of any one of paragraphs T-EE, wherein the AFP-FcRn potentiator enhances binding between Q597 of AFP and E69 of β2m complexed with FcRn.
GG. The pharmaceutical composition of any one of paragraphs T-FF, wherein the AFP-FcRn potentiator enhances binding between E106 of AFP and H161 of FcRn.
HH. The pharmaceutical composition of any one of paragraphs T-GG, wherein the AFP-FcRn potentiator enhances binding between S135 of AFP and H161 of FcRn.
II. The pharmaceutical composition of any one of paragraphs T-HH, wherein the AFP-FcRn potentiator enhances binding between 531, F533, F552, and/or F575 of AFP and W53 of FcRn.
JJ. The pharmaceutical composition of any one of paragraphs T-II, wherein the AFP-FcRn potentiator is an antibody or antigen-binding fragment thereof, a small molecule compound, an RNA or DNA aptamer, or an AFP functional fragment.
KK. The pharmaceutical composition of paragraph JJ, wherein the antibody or antigen-binding fragment thereof is a chimeric, humanized, or completely human antibody or antigen-binding fragment thereof.
LL. The pharmaceutical composition of any one of paragraphs T-KK, wherein the AFP-FcRn potentiator binds FcRn and mimics AFP binding.
MM. The pharmaceutical composition of any one of paragraphs T-LL, wherein the AFP-FcRn potentiator binds or physically interacts with AFP or FcRn, and enhances or promotes interactions between AFP and FcRn.
NN. The pharmaceutical composition of paragraph MM, wherein the AFP-functional fragment comprises Y521 and/or V522 of AFP and can interact with R42 of FcRn.
OO. The pharmaceutical composition of any one of paragraphs JJ or NN, wherein the AFP-functional fragment comprises P492 of AFP and can interact with R69 of FcRn.
PP. The pharmaceutical composition of any one of paragraphs JJ or NN-OO, wherein the AFP-functional fragment comprises Q441 and/or V493 of AFP and can interact with E44 of FcRn.
QQ. The pharmaceutical composition of any one of paragraphsJJ or NN-PP, wherein the AFP-functional fragment comprises H534 and/or E589 of AFP and can interact with N173 of FcRn.
RR. The pharmaceutical composition of any one of paragraphs JJ or NN-QQ, wherein the AFP-functional fragment comprises L484, V493, V497, and/or F512 of AFP and can interact with V57, W59, and/or W61 of FcRn.
SS. The pharmaceutical composition of any one of paragraphs JJ or NN-RR, wherein the AFP-functional fragment comprises T443 of AFP and can interact with E62 and/or W59 of FcRn.
TT. The pharmaceutical composition of any one of paragraphs JJ or NN-SS, wherein the AFP-functional fragment comprises D529 of AFP and can interact with S230 of FcRn.
UU. The pharmaceutical composition of any one of paragraphs JJ or NN-TT, wherein the AFP-functional fragment comprises S527 and/or D528 of AFP and can interact with E50 and/or 67Y of β2m complexed with FcRn.
VV. The pharmaceutical composition of any one of paragraphs JJ or NN-UU, wherein the AFP-functional fragment comprises R604 of AFP and can interact with the carbonyl oxygen at E50 of β2m complexed with FcRn.
WW. The pharmaceutical composition of any one of paragraphs JJ or NN-VV, wherein the AFP-functional fragment comprises Q597 of AFP and can interact with E69 of β2m complexed with FcRn.
XX. The pharmaceutical composition of any one of paragraphs JJ or NN-WW, wherein the AFP-functional fragment comprises E106 of AFP and can interact with H161 of FcRn.
YY. The pharmaceutical composition of any one of paragraphs JJ or NN-XX, wherein the AFP-functional fragment comprises S135 of AFP and can interact with H161 of FcRn.
ZZ. The pharmaceutical composition of any one of paragraphs JJ or NN-YY, wherein the AFP-functional fragment comprises F531, F533, F552, and/or F575 of AFP and can interact with W53 of FcRn.
AAA. A method to inhibit or reduce FcRn and alpha-fetoprotein (AFP) interactions in a disease or disorder associated with elevated AFP levels comprising administering a therapeutically effective amount of a pharmaceutical composition comprising an AFP-FcRn inhibitor of any one of paragraphs A-S to a subject in need thereof.
BBB. The method of paragraph AAA, wherein the subject has or has been diagnosed with cancer.
CCC. The method of any one of paragraphs AAA or BBB, wherein the subject has or has been diagnosed with a cancer or tumor of primitive origin, a tumor of liver origin, such as a hepatoma, a tumor of biliary origin, such as cholangiocarcinoma, stomach cancer, pancreatic cancer, or a teratocarcinoma.
DDD. The method of any one of paragraphs AAA-CCC, further comprising administering an anti-cancer therapy or agent to the subject.
EEE. The method of any one of paragraphs AAA-DDD, further comprising administering a tumor or cancer antigen.
FFF. A method to increase or potentiate FcRn and alpha-fetoprotein (AFP) interactions in diseases or disorders associated with decreased AFP levels or where increasing AFP levels is beneficial comprising administering a therapeutically effective amount of a pharmaceutical composition comprising an AFP-FcRn potentiator of any one of paragraphs T-ZZ to a subject in need thereof. GGG. The method of paragraph FFF, wherein the subject in need is pregnant or is at risk for having a problem with establishing and/or maintaining a pregnancy.
HHH. The method of paragraph FFF, wherein the subject has or has been diagnosed with an autoimmune disease or disorder.
III. The method of paragraph FFF, wherein the subject has or has been diagnosed with host versus graft disease (HVGD), is an organ or tissue transplant recipient, or a recipient of an allogenic transplant.

### EXAMPLES

hAFP transcytosis assays were performed as previously described for IgG. In brief, MDCK II cells expressing hβ₂m and hFcRn or mβ₂m and mFcRn or vector controls (expressing only hβ₂m) were grown to confluence on transwells (Costar) and allowed to polarize over 4 days. 12 hours before the transcytosis experiment, the medium was changed to serum-free media without antibiotics. On the day of experiment, the transwells were incubated with HBSS pH 7.4 for 20 minutes before placing on a new 12 well plate (Costar) where the input chamber contained AFP HBSS pH 6.0 or 7.4 (with) and the exit chamber contained HBSS pH 7.4. For blocking AFP transcytosis with the mouse anti-human FcRn antibody (ADM31) or isotype control (IgG2b), transwells were pre-incubated for 20 min with the respective antibodies in HBSS pH 7.4 prior to the addition of AFP to the same side of the chamber. After 2 hours incubation in 36° C. and 5% CO₂, the medium at the opposite chamber was harvested and the hAFP concentration was measured using ELISA method. For the AFP inhibition of IgG transcytosis, AFP and IgG were both added in HBSS pH 6 to the input chamber and 2 hours later the medium at the opposite chamber was harvested and IgG was quantified using ELISA method.

In vitro cross-presentation assays were carried out by pulsing 1×10⁵ isolated DC with preformed immune complexes (0.5 pg/ml NIP-conjugated OVA+100 μg/ml anti-NIP IgG or anti-NIP IHH-IgG) for 2-3 h followed by extensive washing and the addition of 2×10⁵ purified OT-I CD8⁺ T cells. The presentation assays were carried out accordingly with the distinction of utilizing 2×10⁵ purified OT-II CD4⁺ T cells. Depending on condition, DCs were pre-incubated with hAFP (50 or 100 μg/ml) or HSA (50 or 100 μg/ml) in presence or absence of ADM31 or IgG2b isotype control (75 or 100 μg/ml). Immune complexes were formed using ovalbumin conjugated to the hapten NIP (4-hydroxy-3-iodo-5-nitrophenylacetic acid) and NIP-specific chimeric IgG (IgG) or IHH-IgG. IHH-IgG is a mutational variant of the chimeric IgG protein which contains a NIP-specific mouse Fab fragment and a human IgG1 Fc fragment and which has been rendered incapable of FcRn binding due to the introduction of mutations in three critical amino acids in the Fc region which are required for FcRn ligation. Cytokine secretion was measured after 24 h or 48 h by ELISA. For the measurement of proliferation, OT-I CD8⁺ T cells were stained with eFluor670 Proliferation Dye according to the manufacturer's instructions (eBioscience) and stimulated as described above.

For in vivo enhanced clearance of hIgG upon hAFP administration effect, hFCGRT/hB2M/mFcgrt−/− mice were injected i.p. with hIgG (100 pg/mouse) and the following day with hAFP (100 pg/mouse). 24, 48 and 72 hrs later blood samples were collected and the amount of hIgG was quantified by ELISA and compared to Day 0.

Surface plasmon resonance was conducted using a Biacore 3000 instrument (GE Healthcare) with CM5 sensor chips coupled with recombinant human FcRn & hβ₂m heterodimer protein or mouse FcRn & mβ2m (1000 RU). The coupling was performed by injecting 25 g/mL of the protein diluted in 10 mM sodium acetate pH 5.0 using the amine coupling kit (GE Healthcare). Phosphate buffer (67 mM phosphate buffer, 0.15 M NaCl, 0.005% Tween 20) at pH 6.0 or pH 7.4 was used as running buffer. Glycine pH 2.5 was used for regeneration of the flow cells. hAFP (SinoBiological) was injected at 25° C. with a flow rate of 25 l/min, and data were analyzed using the BIAevaluation 4.1 software where the sensorgrams were zero adjusted and reference cell values subtracted.

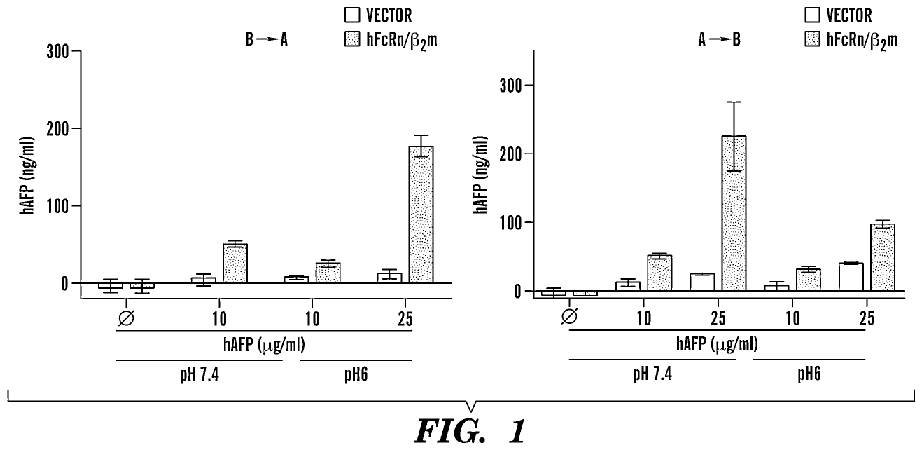

FIG. 1 demonstrates that human AFP (hAFP) is transcytosed by human FcRn (hFcRn) at acidic and neutral pH. Transcytosis of AFP byMDCK II cells co-expressing FcRn and β2microglobulin (hFcRn/β2m) or vector control at pH 6 and 7.4. B→A Basolateral to apical direction, A→B Apical to basolateral direction.

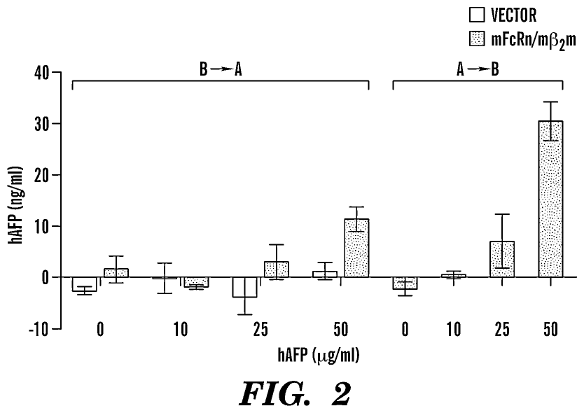

FIG. 2 demonstrates that hAFP is transcytosed by mouse FcRn. Transcytosis of AFP by MDCK II cells co-expressing mouse FcRn and β2microglobulin (mFcRn/mβ2m) or vector control at pH 7.4. B→A Basolateral to apical direction, A→B Apical to basolateral direction.

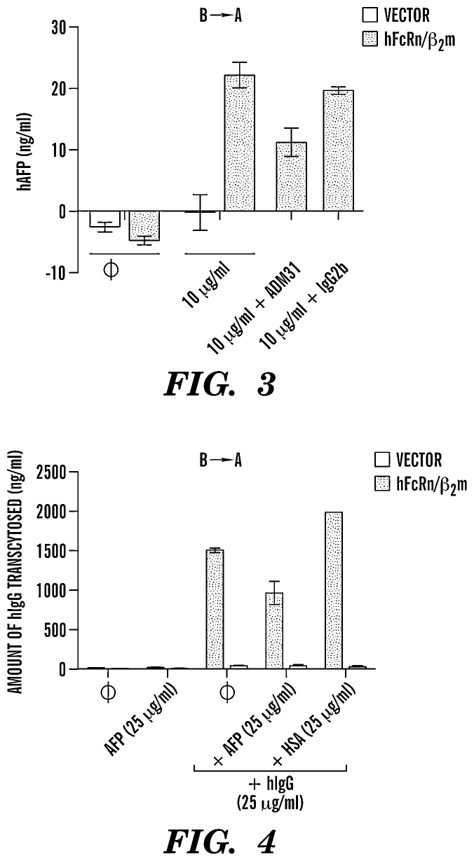

FIG. 3 demonstrates that hAFP transcytosis by hFcRn is blocked by ADM31 antibody. Transcytosis of AFP by MDCK II cells co-expressing hFcRn, hβ2m (mFcRn/mβ2m) or vector control at pH 7.4 in presence of anti-human FcRn antibody (ADM31) or isotype control (IgG2b). B→A Basolateral to apical direction.

FIG. 4 demonstrates that AFP hinders FcRn-mediated transcytosis of IgG. Transcytosis of human IgG by MDCK II cells co-expressing hFcRn, hβ2m, or vector control at pH 6 which have been pre-incubated with hAFP or Human Serum Albumin (HSA) as control at pH 7.4. Basolateral to apical direction is shown.

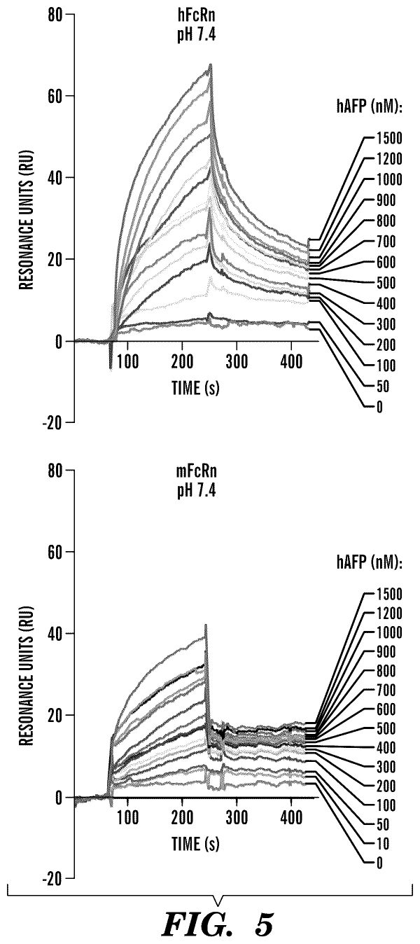

FIG. 5 demonstrates that AFP binds to human and mouse FcRn at neutral pH. SPR analyses of hAFP binding to hFcRn (left panel) or mFcRn (right panel) at neutral pH.

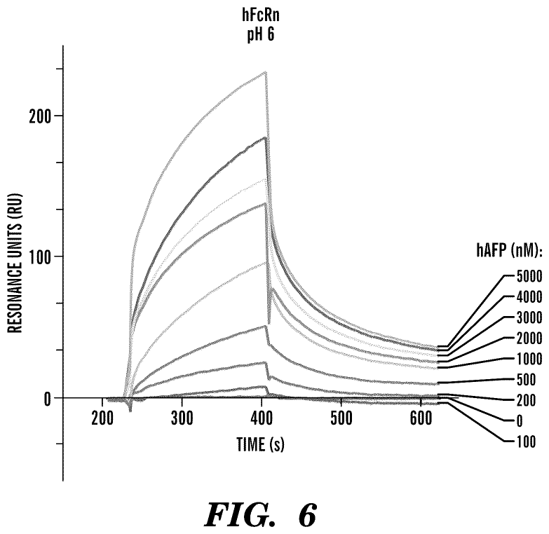

FIG. 6 demonstrates that AFP binds to hFcRn at acidic pH. SPR analyses of hAFP binding to hFcRn at pH 6.

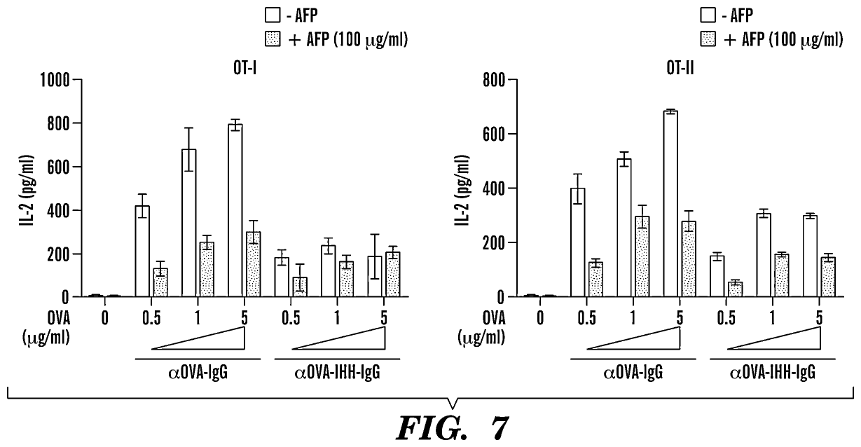

FIG. 7 demonstrates that AFP inhibits T cell stimulation by IgG-IC primed DC. hAFP blocks proliferation (IL-2 secretion) of CD8⁺ (OT-I, left panel) or CD4⁺ (OT-II, right panel) T cells in response to antigen in IgG-IC. BMDC from hFCGRT/hB2M/mFcgrt^{-/-} mice were treated with 100 μg/ml of IgG or IHH-IgG in association with 0, 0.5, 1, or 5 μg/ml of OVA in presence of 100 μg/ml of hAFP and then co-cultured with either OVA-specific CD8⁺ or CD4⁺ T cells. 24 after the stimulation IL-2 secretion in the supernatants were measured by ELISA.

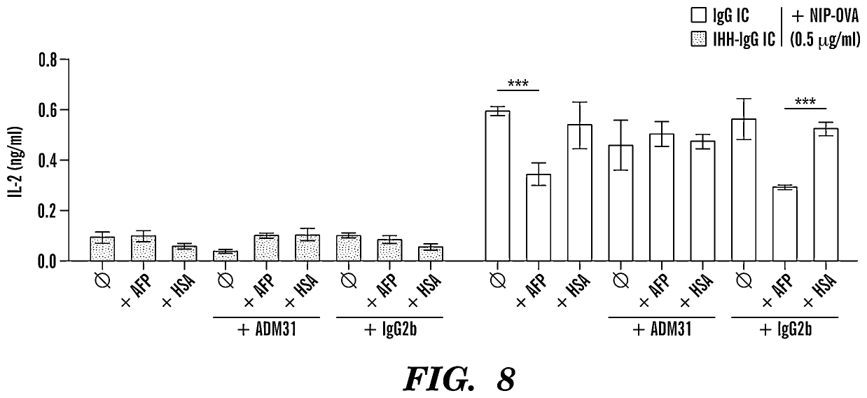

FIG. 8 demonstrates that ADM31 blocks AFP-FcRn-mediated inhibitory functions. ADM31, a monoclonal anti-hFcRn antibody blocks hAFP inhibition of CD8⁺ T cell IL-2 secretion in response to antigen in IgG-IC. BMDC from hFCGRT/hB2M/mFcgrt^{-/-} mice were treated with 100 μg/ml of IgG or IHH-IgG in association with 0.5 μg/ml OVA in presence of 50 μg/ml of hAFP or HSA and 50 μg/ml of ADM31 or isotype control, and then co-cultured with OVA-specific CD8⁺ T cells. 24 after the stimulation IL-2 secretion in the supernatants were measured by ELISA.

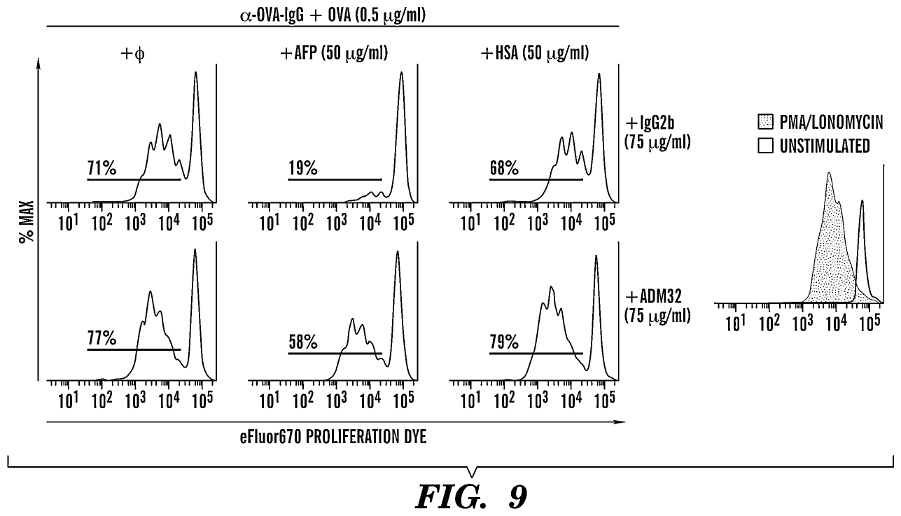

FIG. 9 demonstrates that ADM31 blocks AFP-FcRn-mediated inhibitory functions. ADM31 blocks hAFP inhibition of CD8⁺ T cell proliferation in response to antigen in IgG-IC. BMDC from hFCGRT/hB2M/mFcgrt^{-/-} mice were treated with 100 μg/ml of IgG or IHH-IgG in association with 0.5 g/ml OVA in presence of 50 μg/ml of hAFP or HSA and 50 μg/ml of ADM31 or IgG2b isotype control, and then co-cultured with CD8⁺ T cells labelled with eFluor670 Proliferation Dye. 72 hrs later the cells were acquired. Percent of proliferated cells is displayed.

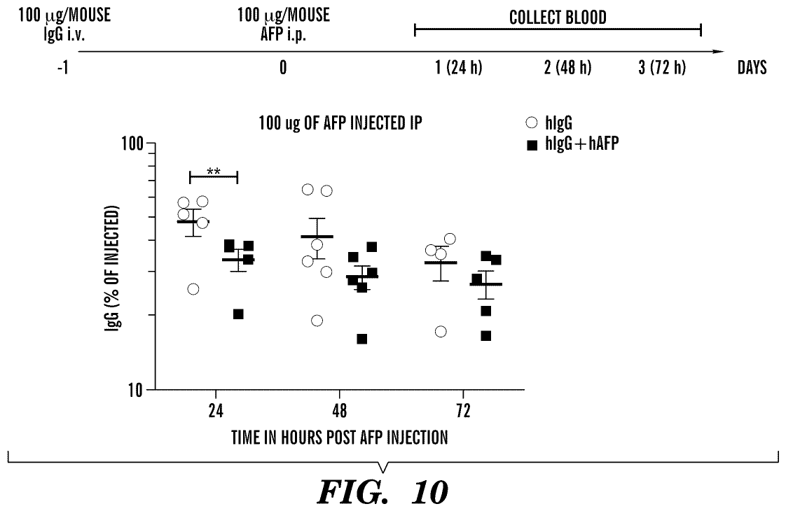

FIG. 10 demonstrates that administration of hAFP results in increased clearance of hIgG antibodies from systemic circulation. hFCGRT/hB2M/mFcgrt^{-/-} mice were injected with hIgG and the following day with hAFP. 24, 48 and 72 hrs later blood samples were collected and the amount of hIgG was quantified by ELISA and compared to Day 0. The results illustrate that AFP injection resulted in faster clearance of hIgG from circulation.

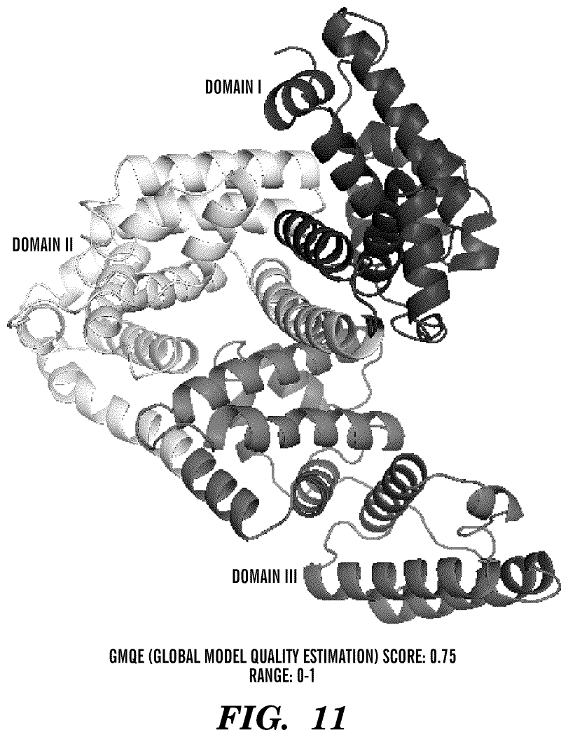

FIG. 11 shows an AFP homology model derived from HSA Crystal Structure (PDB ID: 4N0F).

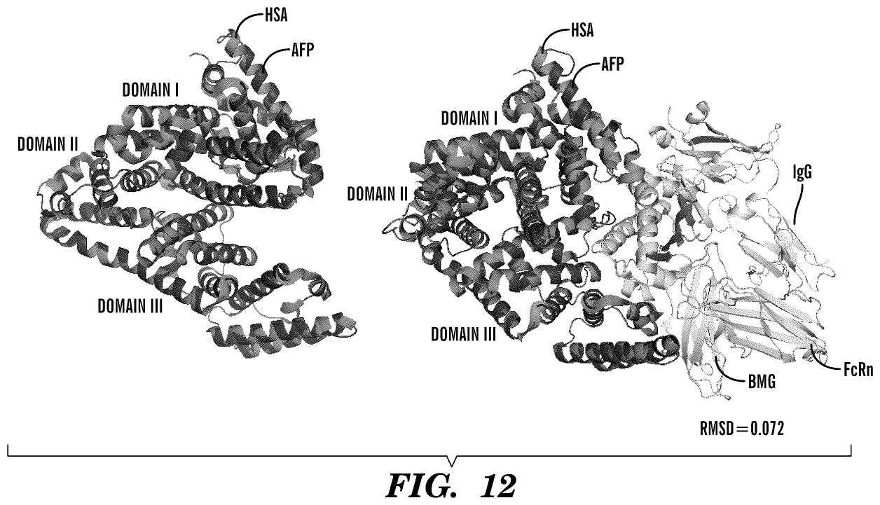

FIG. 12 depicts superimposition of AFP model on FcRn-HSA-IgG ternary complex crystal structure (PDB ID: 4N0U).

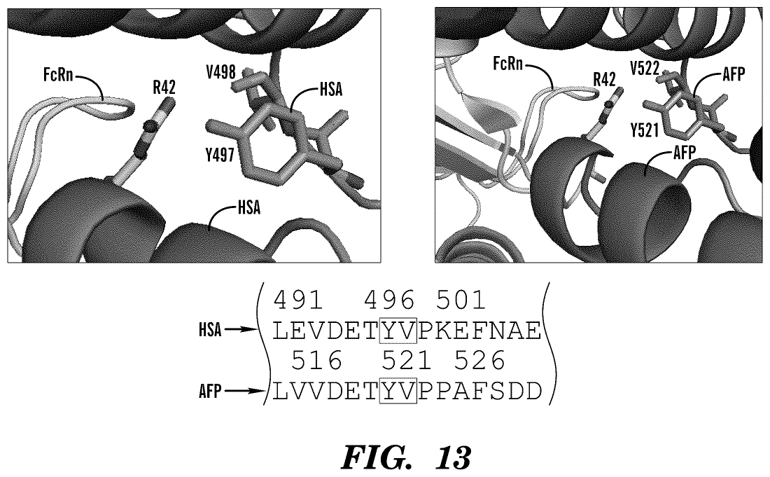

FIG. 13 depicts HSA Y497/V498 residues are conserved in AFP (Y521/V522) and interact with FcRn R42. HSA/AFP conserved residues in Domain III that establish binding to FcRn.

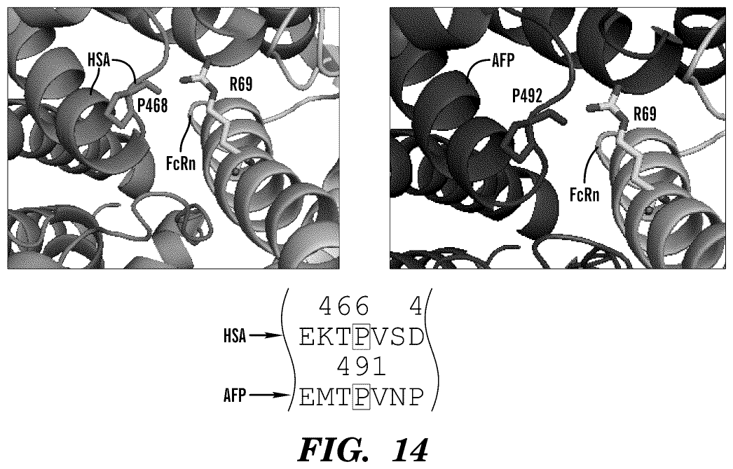

FIG. 14 demonstrates that HSA P468 residue is conserved in AFP (P492) and interacts with FcRn R69. HSA/AFP conserved residues in Domain III that establish binding to FcRn

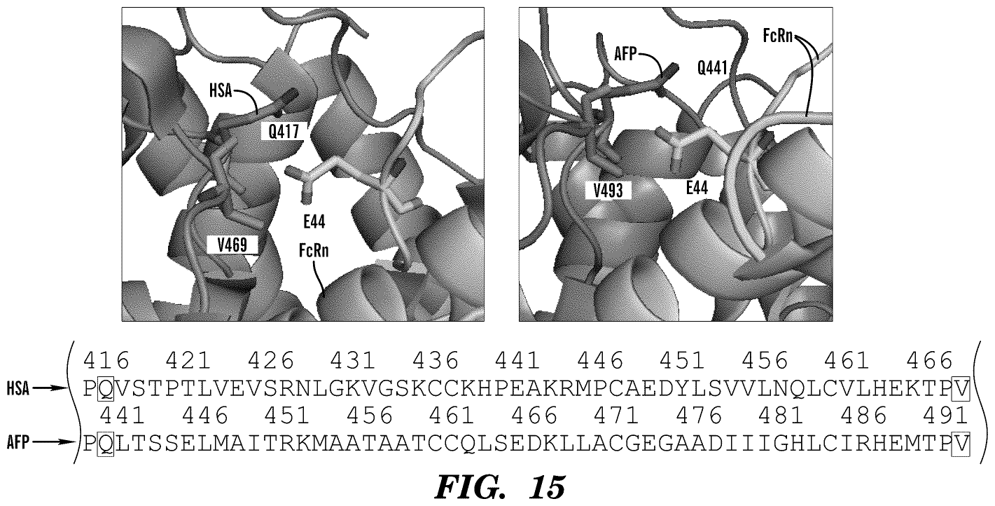

FIG. 15 demonstrates that HSA Q417/V469 residues are conserved in AFP (Q441/V493) and interact with FcRn E44. HSA/AFP conserved residues in Domain III that establish binding to FcRn AFP. HSA V469/AFPV493 make backbone contacts with conserved HSA H464/AFP H488.

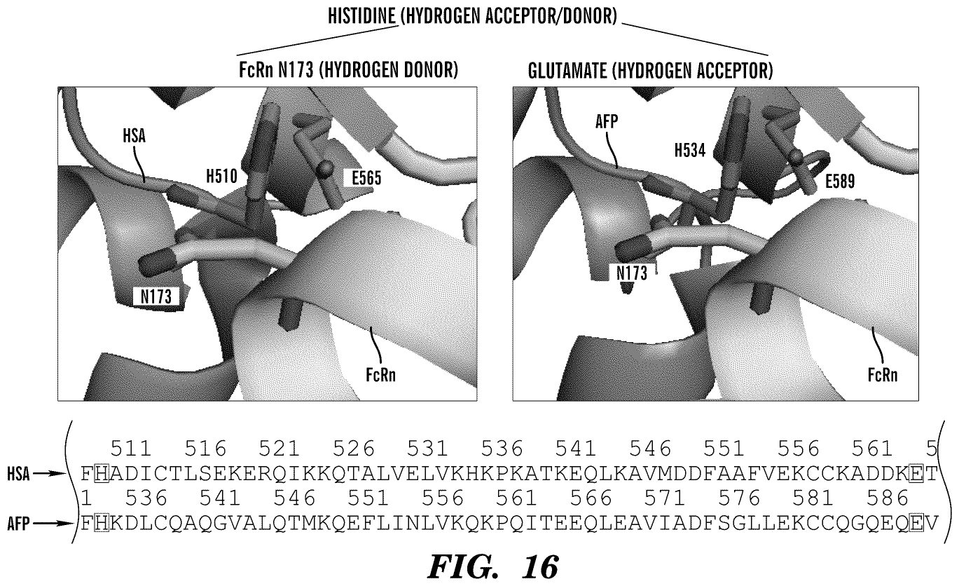

FIG. 16 demonstrates that HSA H510/E565 residues are conserved in AFP (H534/E589) and interact with FcRn N173. HSA/AFP conserved residues in Domain III establish binding to FcRn.

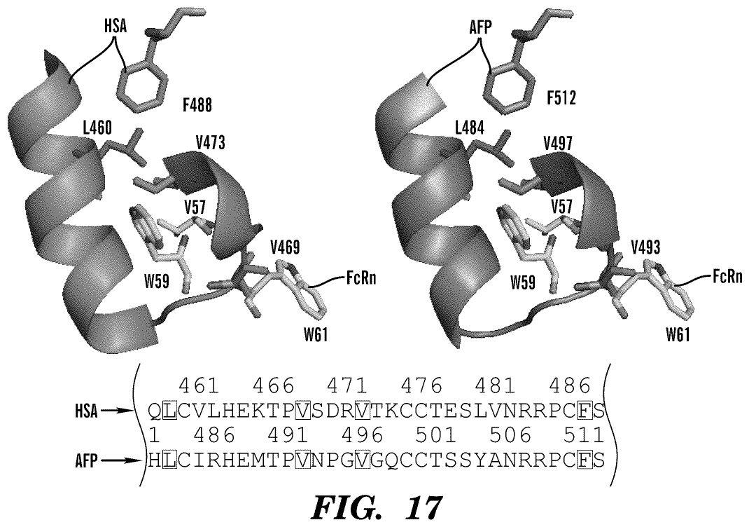

FIG. 17 demonstrates that hydrophobic core centered on HSA L460/V469/V473/F488 is conserved in AFP (L484/V493/V497/F512) and interacts with FcRn V57/W59/W61. HSA/AFP conserved residues in Domain III establish binding to FcRn.

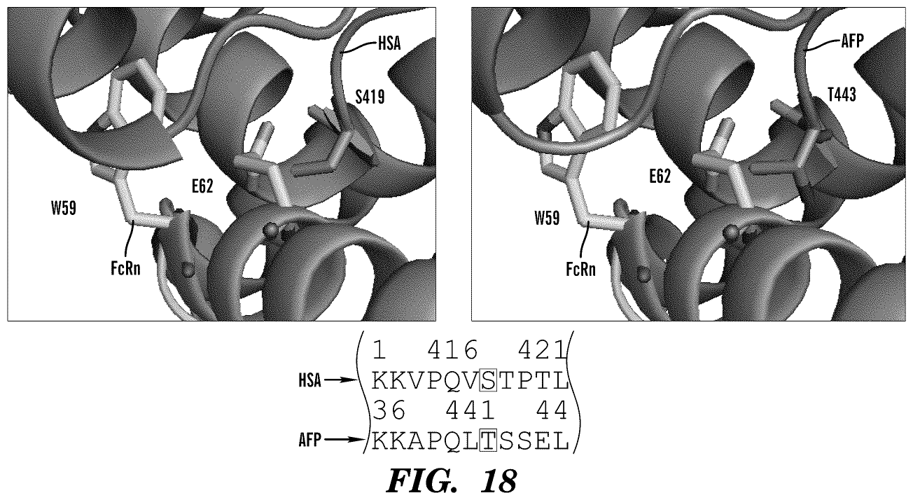

FIG. 18 demonstrates that HSA S419 residue is not conserved in AFP (T443) yet is able to interact with FcRn E62/W59. HSA/AFP non-conserved residues in Domain III preserve AFP binding to FcRn.

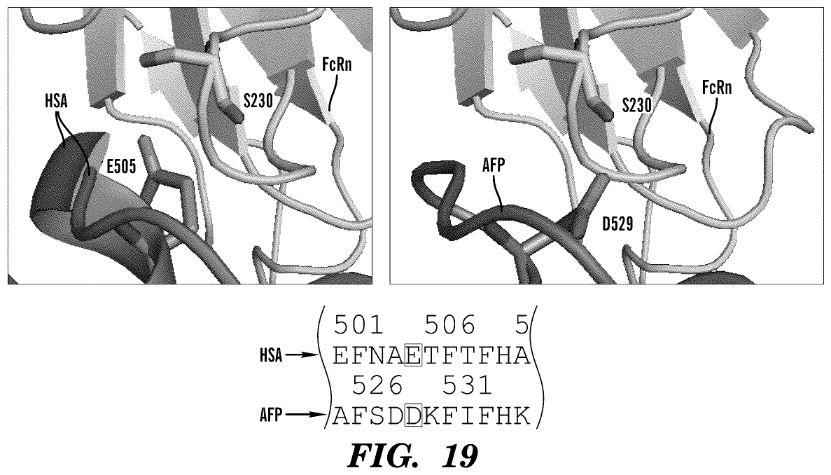

FIG. 19 demonstrates that HSA E505 non-conserved residue in AFP (D529) preserves binding to FcRn S230. HSA/AFP non-conserved residues in Domain III preserve AFP binding to FcRn.

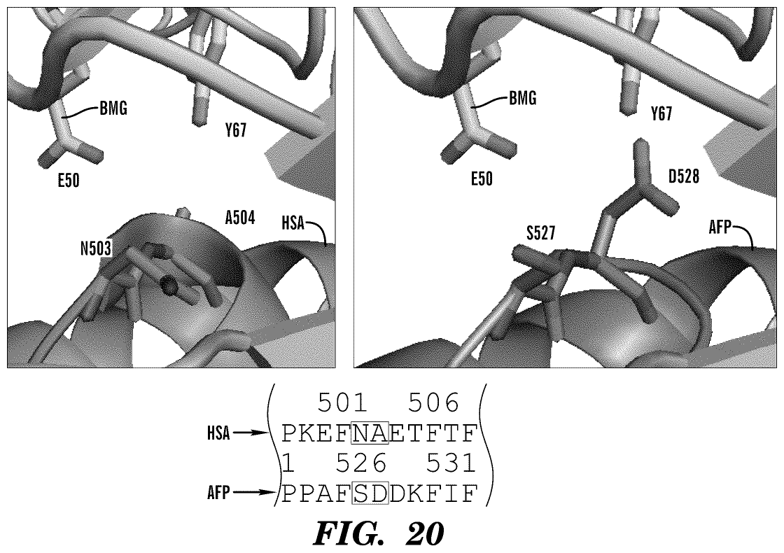

FIG. 20 demonstrates that AFP S527/D528 residues make contacts with β2m E50 and 67Y that are not present in HSA (N503, A504) providing new interactions. HSA/AFP non-conserved residues that increase AFP binding to FcRn through new contacts with β2m and is not pH dependent.

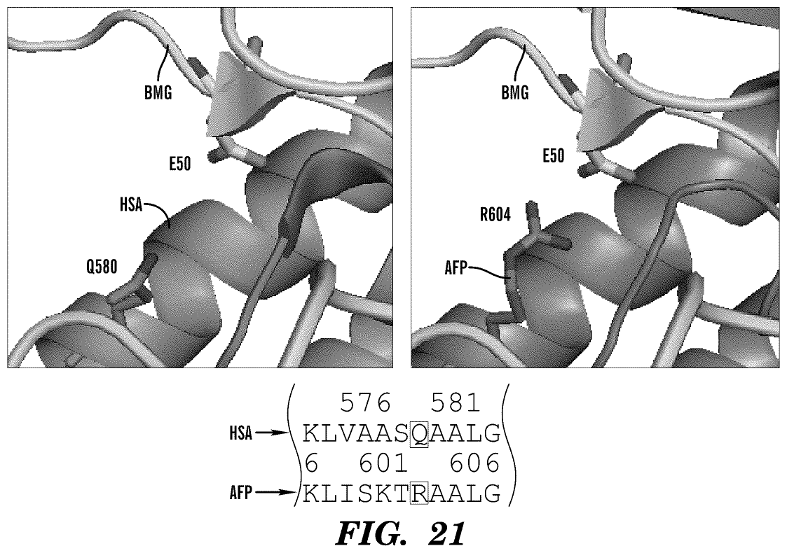

FIG. 21 demonstrates that AFP R604 makes additional contacts with β2m E50, providing new interactions. HSA/AFP non-conserved residues increase AFP binding to β2m. HSA Q580 lacks these interactions.

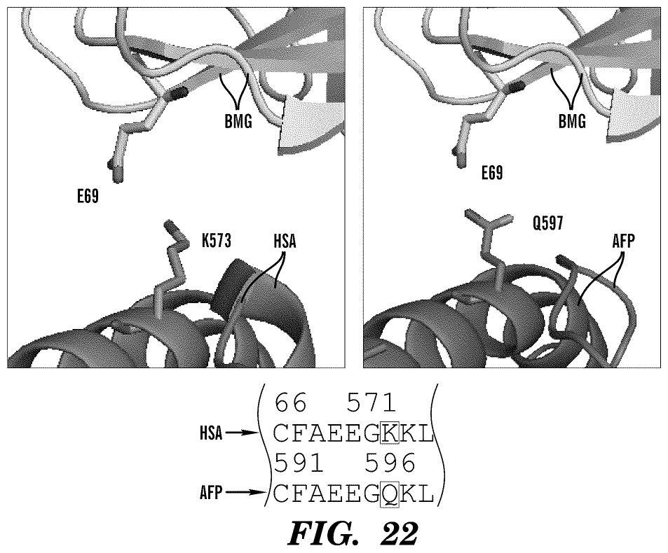

FIG. 22 demonstrates that AFP Q597 residue is better positioned to make contacts with β2m E69 providing stronger interaction. HSA/AFP non-conserved residues establish new and increased AFP-β2m interactions. HSA K573 lacks these interactions.

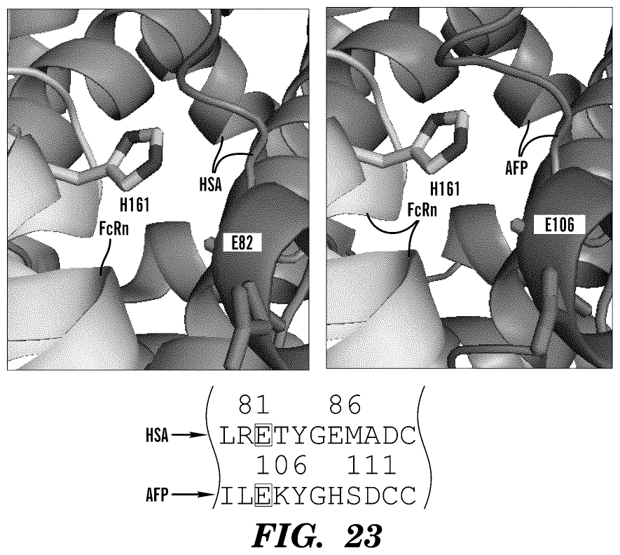

FIG. 23 demonstrates that AFP (E106) conserved residue (with HSA E82) makes long range interaction with FcRn H161. Conserved HSA/AFP residues in Domain I interact with FcRn.

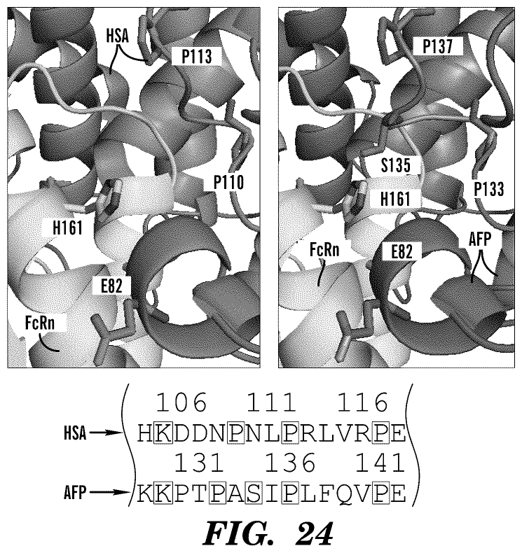

FIG. 24 demonstrates that AFP S135 allows AFP interface to come closer to FcRn and makes ˜3 Å interactions with FcRn H161, which is absent in HSA. AFP Domain I-FcRn interaction suggests neutral pH binding. Nearby conserved proline in HSA/AFP occupy same space in interface.

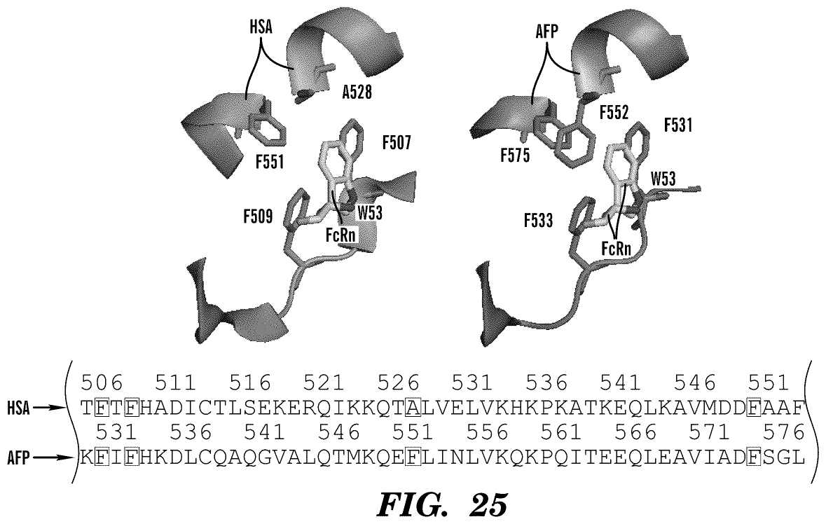

FIG. 25 demonstrates a larger hydrophobic core in AFP (F531/F533/F552/F575) centered on FcRn W53: AFP F552 results in stronger AFP-FcRn interactions than HSA A528. AFP-FcRn interactions indicates neutral pH binding AFP (F531/F533/F552/F575) versus HSA (F507/F509/A528/F551).

TABLE 1
SUMMARY OF INTERACTIONS OF AFP WITH FCRN

| HSA | AFP | FcRn Contact | Interaction |
|---|---|---|---|
| Residues | Residues | Residues | change |
| HSA/AFP Conserved Interactions with FcRn | | | |
| Y497 | Y521 | R42 | = |
| V498 | V522 | | |
| P468 | P492 | R69 | = |
| Q417 | Q441 | E44 | = |
| V469 | V493 | | |
| H464 | H488 | | |
| H510 | H534 | N173 | = |
| E565 | E589 | | |
| L460 | L484 | V57 | = |
| V469 | V493 | W59 | |
| V473 | V497 | W61 | |
| F488 | F512 | | |
| D89 | D112 | N149 | = |
| E82 | E106 | H161 | = |
| S419 | T443 | E62, W59 | = |
| E505 | D529 | S230 | = |
| Novel AFP Interactions With FcRn | | | |
| — | S135 | H161 | ↑ Binding |
| F507 | F531 | W53 | ↑ Binding |
| F509 | F533 | | |
| F551 | F575 | | |
| A528 | F552 | | |
| P421 | S445 | Q56 | ↑ Binding |
| Novel AFP Interactions with Beta-2-Microglobulin | | | |
| HSA | AFP | β₂-Microglobulin | |
| Residues | Residues | Residues | |
| N503 | S527 | E50 | ↑ Binding |
| A504 | D528 | Y67 | |
| Q580 | R604 | | |
| K573 | Q597 | E69 | ↑ Binding |

What is claimed is:

1. A pharmaceutical composition comprising an inhibitor of AFP-FcRn and a pharmaceutically acceptable carrier, wherein said inhibitor of AFP-FcRn inhibits binding between alpha-fetoprotein (AFP) and FcRn.

2. The pharmaceutical composition of claim 1, wherein the inhibitor of AFP-FcRn comprises a T451I and/or D536V polymorphism of wild-type AFP.

3. The pharmaceutical composition of claim 1, wherein the inhibitor of AFP-FcRn inhibits binding between:
(a) Y521 and/or V522 of AFP and R42 of FcRn;
(b) P492 of AFP and R69 of FcRn;
(c) Q441 and/or V493 of AFP and E44 of FcRn;
(d) H534 and/or E589 of AFP and N173 of FcRn;
(e) the hydrophobic core of AFP and FcRn;
(f) L484, V493, V497, and/or F512 of AFP and V57, W59, and/or W61 of FcRn;
(g) T443 of AFP and E62 and/or W59 of FcRn;
(h) D529 of AFP and S230 of FcRn;
(i) S527 and/or D528 of AFP and E50 and/or 67Y of β2m complexed with FcRn;
(i) R604 of AFP and the carbonyl oxygen at E50 of β2m complexed with FcRn;
(k) Q597 of AFP and E69 of β2m complexed with FcRn;
(l) E106 of AFP and H161 of FcRn;
(m) S135 of AFP and H161 of FcRn; or
(n) F531, F533, F552, and/or F575 of AFP and W53 of FcRn.

4-16. (canceled)

17. The pharmaceutical composition of claim 1, wherein the inhibitor of AFP-FcRn is an antibody or antigen-binding fragment thereof, a small molecule compound, or an RNA or DNA aptamer.

18. The pharmaceutical composition of claim 17, wherein the antibody or antigen-binding fragment thereof is a chimeric, humanized, or completely human antibody or antigen-binding fragment thereof.

19. The pharmaceutical composition of claim 1, wherein the inhibitor of AFP-FcRn inhibits or blocks the AFP binding site on FcRn.

20. A pharmaceutical composition comprising an AFP-FcRn potentiator and a pharmaceutically acceptable carrier.

21. The pharmaceutical composition of claim 20, wherein the AFP-FcRn potentiator comprises a G109R, R487S, and/or S445L polymorphism of wild-type alpha-fetoprotein (AFP) that increases AFP-FcRn binding.

22. The pharmaceutical composition of claim 20, wherein the AFP-FcRn potentiator enhances binding between:
(a) Y521 and/or V522 of AFP and R42 of FcRn;
(b) P492 of AFP and R69 of FcRn;
(c) Q441 and/or V493 of AFP and E44 of FcRn;
(d) H534 and/or E589 of AFP and N173 of FcRn;
(e) the hydrophobic core of AFP and FcRn;
(f) L484, V493, V497, and/or F512 of AFP and V57, W59, and/or W61 of FcRn;
(g) T443 of AFP and E62 and/or W59 of FcRn;
(h) D529 of AFP and S230 of FcRn;
(i) S527 and/or D528 of AFP and E50 and/or 67Y of β2m complexed with FcRn;
(i) R604 of AFP and the carbonyl oxygen at E50 of β2m complexed with FcRn;
(k) Q597 of AFP and E69 of β2m complexed with FcRn;
(l) E106 of AFP and H161 of FcRn;
(m) S135 of AFP and H161 of FcRn; or
(n) F531, F533, F552, and/or F575 of AFP and W53 of FcRn.

23-35. (canceled)

36. The pharmaceutical composition of claim 20, wherein the AFP-FcRn potentiator is an antibody or antigen-binding fragment thereof, a small molecule compound, an RNA or DNA aptamer, or an AFP functional fragment.

37. The pharmaceutical composition of claim 36, wherein the antibody or antigen-binding fragment thereof is a chimeric, humanized, or completely human antibody or antigen-binding fragment thereof.

38. The pharmaceutical composition of claim 20, wherein the AFP-FcRn potentiator binds FcRn and mimics AFP binding.

39. The pharmaceutical composition of claim 20, wherein the AFP-FcRn potentiator binds or physically interacts with AFP or FcRn, and enhances or promotes interactions between AFP and FcRn.

40. The pharmaceutical composition of claim 36, wherein the AFP-functional fragment comprises:
(a) Y521 and/or V522 of AFP and can interact with R42 of FcRn;
(b) P492 of AFP and can interact with R69 of FcRn;
(c) Q441 and/or V493 of AFP and can interact with E44 of FcRn;
(d) H534 and/or E589 of AFP and can interact with N173 of FcRn;
(e) L484, V493, V497, and/or F512 of AFP and can interact with V57, W59, and/or W61 of FcRn;
(f) T443 of AFP and can interact with E62 and/or W59 of FcRn;
(g) D529 of AFP and can interact with S230 of FcRn;
(h) S527 and/or D528 of AFP and can interact with E50 and/or 67Y of β2m complexed with FcRn;
(i) R604 of AFP and can interact with the carbonyl oxygen at E50 of β2m complexed with FcRn;
(i) Q597 of AFP and can interact with E69 of β2m complexed with FcRn;
(k) E106 of AFP and can interact with H161 of FcRn;
(l) S135 of AFP and can interact with H161 of FcRn; or
(m) F531, F533, F552, and/or F575 of AFP and can interact with W53 of FcRn.

41-52. (canceled)

53. A method to inhibit or reduce FcRn and alpha-fetoprotein (AFP) interactions in a disease or disorder associated with elevated AFP levels comprising administering a therapeutically effective amount of a pharmaceutical composition comprising an AFP-FcRn inhibitor of claim 1 to a subject in need thereof.

54. The method of claim 53, wherein the subject has or has been diagnosed with cancer; or wherein the subject has or has been diagnosed with a cancer or tumor of primitive origin, a tumor of liver origin, such as a hepatoma, a tumor of biliary origin, such as cholangiocarcinoma, stomach cancer, pancreatic cancer, or a teratocarcinoma.

55. (canceled)

56. The method of claim 53, further comprising administering an anti-cancer therapy or agent to the subject.

57. The method of claim 53, further comprising administering a tumor or cancer antigen.

58. A method to increase or potentiate FcRn and alpha-fetoprotein (AFP) interactions in diseases or disorders associated with decreased AFP levels or where increasing AFP levels is beneficial comprising administering a therapeutically effective amount of a pharmaceutical composition comprising an AFP-FcRn potentiator of claim 20 to a subject in need thereof.

59. The method of claim 58, wherein the subject in need is pregnant or is at risk for having a problem with establishing and/or maintaining a pregnancy: wherein the subject has or has been diagnosed with an autoimmune disease or disorder: or wherein the subject has or has been diagnosed with host versus graft disease (HVGD), is an organ or tissue transplant recipient, or a recipient of an allogenic transplant.

60-61. (canceled)


SEQUENCE LISTING (31 sequences)

SEQ ID NO: 1 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 2 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 4 - protein, 575 aa, synthetic construct
  REGION 1..575: note = Description of Artificial Sequence: Synthetic polypeptide
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADF

SEQ ID NO: 5 - protein, 92 aa, synthetic construct
  REGION 1..92: note = Description of Artificial Sequence: Synthetic polypeptide
LCIRHEMTPVNPGVGQCCTSSYANRRPCFSSLVVDETYVPPAFSDDKFIFHKDLCQAQGVALQTMKQEFLINLVKQKPQITEEQLEAVIADF

SEQ ID NO: 6 - protein, 15 aa, Homo sapiens
LEVDETYVPKEFNAE

SEQ ID NO: 7 - protein, 15 aa, Homo sapiens
LVVDETYVPPAFSDD

SEQ ID NO: 8 - protein, 7 aa, Homo sapiens
EKTPVSD

SEQ ID NO: 9 - protein, 7 aa, Homo sapiens
EMTPVNP

SEQ ID NO: 10 - protein, 54 aa, Homo sapiens
PQVSTPTLVEVSRNLGKVGSKCCKHPEAKRMPCAEDYLSVVLNQLCVLHEKTPV

SEQ ID NO: 11 - protein, 54 aa, Homo sapiens
PQLTSSELMAITRKMAATAATCCQLSEDKLLACGEGAADIIIGHLCIRHEMTPV

SEQ ID NO: 12 - protein, 58 aa, Homo sapiens
FHADICTLSEKERQIKKQTALVELVKHKPKATKEQLKAVMDDFAAFVEKCCKADDKET

SEQ ID NO: 13 - protein, 58 aa, Homo sapiens
FHKDLCQAQGVALQTMKQEFLINLVKQKPQITEEQLEAVIADFSGLLEKCCQGQEQEV

SEQ ID NO: 14 - protein, 31 aa, Homo sapiens
QLCVLHEKTPVSDRVTKCCTESLVNRRPCFS

SEQ ID NO: 15 - protein, 31 aa, Homo sapiens
HLCIRHEMTPVNPGVGQCCTSSYANRRPCFS

SEQ ID NO: 16 - protein, 11 aa, Homo sapiens
KKVPQVSTPTL

SEQ ID NO: 17 - protein, 11 aa, Homo sapiens
KKAPQLTSSEL

SEQ ID NO: 18 - protein, 11 aa, Homo sapiens
EFNAETFTFHA

SEQ ID NO: 19 - protein, 11 aa, Homo sapiens
AFSDDKFIFHK

SEQ ID NO: 20 - protein, 11 aa, Homo sapiens
PKEFNAETFTF

SEQ ID NO: 21 - protein, 11 aa, Homo sapiens
PPAFSDDKFIF

SEQ ID NO: 22 - protein, 11 aa, Homo sapiens
KLVAASQAALG

SEQ ID NO: 23 - protein, 11 aa, Homo sapiens
KLISKTRAALG

SEQ ID NO: 24 - protein, 9 aa, Homo sapiens
CFAEEGKKL

SEQ ID NO: 25 - protein, 9 aa, Homo sapiens
CFAEEGQKL

SEQ ID NO: 26 - protein, 11 aa, Homo sapiens
LRETYGEMADC

SEQ ID NO: 27 - protein, 11 aa, Homo sapiens
ILEKYGHSDCC

SEQ ID NO: 28 - protein, 15 aa, Homo sapiens
HKDDNPNLPRLVRPE

SEQ ID NO: 29 - protein, 15 aa, Homo sapiens
KKPTPASIPLFQVPE

SEQ ID NO: 30 - protein, 49 aa, Homo sapiens
TFTFHADICTLSEKERQIKKQTALVELVKHKPKATKEQLKAVMDDFAAF

SEQ ID NO: 31 - protein, 49 aa, Homo sapiens
KFIFHKDLCQAQGVALQTMKQEFLINLVKQKPQITEEQLEAVIADFSGL